(19) United States
(12) Patent Application Publication
(10) Pub. No.: US 2024/0074804 A1
(43) Pub. Date: Mar. 7, 2024

(54) DEVICE AND METHOD FOR ELECTROPORATION BASED TREATMENT OF STENOSIS OF A TUBULAR BODY PART

(71) Applicant: Virginia Tech Intellectual Properties, Inc., Blacksburg, VA (US)

(72) Inventors: Robert E. Neal, Richmond, VA (US); Paulo A. Garcia, Somerville, MA (US); Rafael V. Davalos, Aptos, CA (US); Peter Callas, Castro Valley, CA (US)

(21) Appl. No.: 18/502,967

(22) Filed: Nov. 6, 2023

Related U.S. Application Data
(63) Continuation of application No. 16/865,772, filed on May 4, 2020.
Application No. 16/865,772 is a continuation of application No. 13/550,307, filed on Jul. 16, 2012, now Pat. No. 10,702,326.
(60) Provisional application No. 61/508,251, filed on Jul. 15, 2011.

(51) Int. Cl. A61B 18/00; A61B 18/14

(52) U.S. Cl. CPC A61B 18/00; A61B 18/1492; A61B 2018/0022; A61B 2018/00613; A61B 2018/00767; A61B 2018/00875; A61B 2018/1467; A61B 2090/3966

(57) ABSTRACT

The present invention relates to medical devices and methods for treating a lesion such as a vascular stenosis using non-thermal irreversible electroporation (NTIRE). Embodiments of the present invention provide a balloon catheter type NTIRE device for treating a target lesion comprising a plurality of electrodes positioned along the balloon that are electrically independent from each other so as to be individually selectable in order to more precisely treat an asymmetrical lesion in which the lesion extends only partially around the vessel.

### CROSS-REFERENCE TO RELATED APPLICATIONS

This application is a Continuation application of U.S. patent application Ser. No. 13/550,307, filed Jul. 16, 2012, which relies on the disclosure of and claims priority to and the benefit of the filing date of U.S. Provisional Application No. 61/508,251, filed Jul. 15, 2011. The disclosures of which are hereby incorporated by reference herein in their entireties.

### BACKGROUND OF THE INVENTION

### Field of the Invention

The present invention relates to medical devices and methods for treating, reducing, or preventing stenosis using non-thermal irreversible electroporation. Embodiments of the present invention provide balloon catheter devices for treating or preventing stenosis comprising a plurality of electrodes for selectively and irreversibly electroporating a portion of the inner circumference of a tubular structure within the body. Such devices, systems and methods are particularly useful for treating asymmetrical stenosis.

### Description of Related Art

Atherosclerosis is the main cause of heart attack, stroke and gangrene of the extremities. See Burt H M, Hunter W L (2006), Drug-eluting stents: a multidisciplinary success story, Adv Drug Deliv Rev 58: 350-357 (“Burt 2006”); and Lusis A J (2000) Atherosclerosis, Nature 407: 233-241 (“Lusis 2000”). Three different processes have been identified in studies of animals with induced hypercholesterolaemia that are thought to participate in the formation of atherosclerotic lesions: 1) proliferation of smooth muscle cells, macrophages and lymphocytes; 2) the formation by smooth muscle cells of a connective tissue matrix comprising elastic fiber proteins, collagen and proteoglycans; and 3) accumulation of lipid and mostly free and sterified cholesterol in the surrounding matrix and the associated cells. See Ross R (1993), The pathogenesis of atherosclerosis: a perspective for the 1990s, Nature 362: 801-809.

The introductions of balloon angioplasty and stent implantation in the coronary arteries have reduced significantly the fatalities associated with this disease, however, coronary artery restenosis and neointimal hyperplasia remain clinical problems. See Lusis 2000; and A I Suwaidi J, Berger P B, Holmes D R, Jr. (2000) Coronary artery stents, Jama 284: 1828-1836. Millions of people are affected by atherosclerosis. One feature of this disease is stenosis, which is defined as an abnormal narrowing or contraction of a tubular body part such as arteries, veins, non-vascular ducts and other tubular structures such as urethra, fallopian tubes, esophageal, bronchial passages, and the like. Stenosis causes decreased blood flow through the vessel. A common treatment for stenosis is bypass surgery with less invasive procedures, such as angioplasty procedures like PTA (percutaneous transluminal angioplasty) also available. Angioplasty involves inserting a balloon catheter into the body to the location of the stenosis, then inflating the balloon against the lesion, and applying pressure to compress the lesion and widen or restore the inside diameter of the blood vessel to restore blood flow. Variations of PTA procedures have been used to treat peripheral arterial stenosis, coronary lesions and other non-vascular tubular structures such as biliary ducts.

Although PTA treatments find success in restoring blood flow, such success may be limited or temporary under certain circumstances. For instance, it has been found that anywhere from three to six months following the angioplasty procedure about half of those treated with PTA develop a re-narrowing or occlusion of the vessel, referred to as restenosis. While the original blockage is formed by plaque deposits on the vessel wall, restenosis is caused by growth of smooth muscle cells of the treated artery after angioplasty. It is the trauma imposed on the vessel wall during angioplasty itself that is the cause of restenosis. More particularly, the body reacts to the angioplasty procedure as an injury and produces scar tissue as cells regenerate on the inner wall of the blood vessel in response to the procedure. It is overgrowth of these cells that causes the restenosis, which is the recurrence of stenosis after the PTA procedure. A second angioplasty procedure or bypass are common treatments for restenosis, but each of these exposes the patient to additional risks. This is because the angioplasty procedure is often a temporary fix as it will retraumatize the vessel wall—resulting in the recurrence of smooth muscle cell proliferation. Adding further complexity to the issue, restenosis often presents itself asymmetrically, characterized by cellular regrowth on only portions of the circumference of the vessel wall. It has been found that eccentric and polypoid narrowings are not amenable to treatment with PTA alone. See Becker G J, Katzen B T, Dake M D, Noncoronary angioplasty, Radiology 1989; 170:921-940.

In attempts to limit the amount of restenosis after angioplasty, efforts have been made to reduce the trauma associated during treatment procedures for stenosis. Such efforts include using balloon catheters equipped for cutting or excising the lesions or in combination with an endomyocardial biopsy device. These efforts, however, have not proven any greater success over conventional angioplasty techniques in preventing restenosis after surgery.

Post-angioplasty approaches for reducing restenosis have also been pursued. One such technique involves implanting drug-eluting stents comprising compositions for suppressing the growth of scar tissue. These techniques have been known to reduce restenosis but are not preferred due to complications, such as localized blood clots after elution of the drug, stent fracture, or other long-term implant issues. Most notable risk factors with stents concern the arterial wall injury that is generated with the implantation of the stent and the pressure applied by the balloon. In-stent restenosis after bare-metal stent (BMS) placement results in an aggressive healing response (neointimal hyperplasia) that causes vascular narrowing. See Burt 2006; Legrand V (2007), Therapy insight: diabetes and drug-eluting stents, Nat Clin Pract Cardiovasc Med 4: 143-150; and Ward M R, Pasterkamp G, Yeung A C, Borst C (2000) Arterial remodeling, Mechanisms and clinical implications, Circulation 102: 1186-1191.

Still others have used angioplasty combined with a technique referred to as non-thermal irreversible electroporation. The IRE approach generally involves treatment of the cells subjected to angioplasty to a therapeutic electric field. The goal is to target the vascular cells to ablate and kill the cells without causing thermal or mechanical damage. This approach selectively kills the target cells while avoiding damage to the structure of the artery and surrounding tissue. Restenosis is thus avoided or reduced because the targeted vascular cells are killed, which then do not have the capability of forming scar tissue (neointimal).

Generally, irreversible electroporation (IRE) is a minimally invasive technique to ablate undesired tissue. See Davalos R V, Mir L M, Rubinsky B (2005), Tissue ablation with irreversible electroporation, Annals of Biomedical Engineering 33: 223-231 (“Davalos 2005”). Maor and colleagues showed that IRE reduces the vascular smooth muscle cells population of major blood vessels without affecting the extracellular matrix, which is crucial in the treatment of coronary artery disease. See Maor E, Ivorra A, Leor J, Rubinsky B (2007), The effect of irreversible electroporation on blood vessels, Technology in Cancer Research and Treatment 6: 307-312. The procedure involves delivering a series of low energy (intense but short) electric pulses to the targeted tissue. These pulses permanently destabilize the cell membranes of the treated tissue and cause cell death. IRE has been shown to be an effective means of tissue ablation that does not require drugs, and creates no secondary thermal effects thereby, preserves extracellular matrix, micro-vasculature and nerves. See Rubinsky B (2007), Irreversible Electroporation in Medicine, Technology in Cancer Research and Treatment 6: 255-260. Furthermore, IRE ablates tissue with sub-millimeter resolution and the treated area can be imaged in real-time using ultrasound, or other imaging techniques such as Magnetic Resonance Imaging, Computed Tomography and/or Intravascular Ultrasound (IVUS).

More particularly, as a result of being exposed to the IRE electric field, the pores of the target cells are opened to a degree beyond which they can recover and the cells die. Concerning restenosis in particular, with fewer cells remaining on the vascular wall after the angioplasty procedure, the cells are unable to grow thus preventing restenosis altogether, or the cells which are limited in number can only experience a limited amount of cellular regrowth thus reducing the amount of restenosis. IRE can be performed before, during, and/or after angioplasty. In some cases, the IRE is preferably performed before restenosis occurs, e.g., before angioplasty to treat tissue that will later be exposed to an angioplasty procedure.

It has been known to use IRE on blood vessels using plate electrodes placed around the carotid artery to apply the electric pulses. See Maor, E., A. Ivorra, J. Leor, and B. Rubinsky, The Effect of Irreversible Electroporation on Blood Vessels, Technol Cancer Res Treat, 2007, 6(4): p. 307-312; Maor, E., A. Ivorra, J. Leor, and B. Rubinsky, Irreversible electroporation attenuates neointimal formation after angioplasty, IEEE Trans Biomed Eng, 2008, 55(9): p. 2268-74; and Maor, E., A. Ivorra, and B. Rubinsky, Non Thermal Irreversible Electroporation: Novel Technology for Vascular Smooth Muscle Cells Ablation, PLoS ONE, 2009, 4(3): p. e4757. Unfortunately, this electrode design is highly invasive and requires the physical exposure of the targeted vessel in order to treat it.

In other existing IRE procedures for treatment of restenosis, the entire circumference of the vessel wall is exposed to the IRE electric field. In such designs it has been known to use an electrode with positive and negative independent conducting surfaces, which are energized in an all-or-nothing system, energizing the entire circumference of the electrode at the same time and with equal energy delivery. Such an approach is not desirable for cases of asymmetrical restenosis, however, where only a portion or less than the entire circumference of the vessel wall is diseased. In treating asymmetric restenosis with circumferential IRE, vascular cells on non-diseased portions of the vessel wall are unnecessarily destroyed.

Thus, it is apparent that there is a need for less invasive, less traumatic treatment procedures for treating, reducing, or preventing restenosis. Especially needed are procedures capable of targeting only the diseased portions of the vascular structure, or capable of targeting only portions of the vascular structure susceptible to restenosis, such as tissue previously subjected to stenosis treatment and/or stenotic tissue prior to treatment.

### SUMMARY OF THE INVENTION

To this end, embodiments of the present invention provide devices, systems, and methods for treating lesions such as vascular stenosis including restenosis. Especially preferred are such devices, systems, and methods for treating asymmetric lesions, i.e., lesions that extends only partially around the vessel.

Non-thermal irreversible electroporation (NTIRE) treatment methods and devices of the invention include a plurality of electrodes positioned along a balloon of a balloon catheter that are electrically independent from each other so as to be individually selectable in order to more precisely treat an asymmetrical lesion.

According to one aspect of the present invention, a method of treating a stenosis of a tubular body part by non-thermal irreversible electroporation is provided. The method involves: inserting, through the tubular body part, a balloon catheter having at least three electrodes positioned and spaced apart along the balloon, the electrodes being electrically independent from each other; expanding the balloon to bring the electrodes near a stenosis to be treated; determining which electrodes are near the stenosis; and applying electrical pulses to the electrodes according to the determination of which electrodes are near the stenosis, the applied pulses being in an amount which is sufficient to induce irreversible electroporation of cells of the stenosis, but which is insufficient to induce thermal damage to substantially all of the cells of the stenosis such that substantially all stenosis cells are killed by non-thermal irreversible electroporation.

In another aspect of the invention, an entire circumferential area of a vessel can be treated by selectively energizing selected conductive surfaces of an electrode, i.e., delivering the electrical charge asymmetrically with respect to the vessel, however, selection of the conductive surfaces can be rotated for example sequentially to cover a whole circumferential section of a vessel. In such embodiments, since smaller segments of the electrode are being activated at certain times, less power is needed and developing electronics for this would be less complex task.

In another aspect of the invention, the step of applying electrical pulses includes selecting at least one electrode to which the electrical pulses are not to be applied.

In another aspect of the invention, the step of applying electrical pulses includes connecting through a switch a pulse generator output to any pair of the electrodes independent of the other electrodes.

In another aspect of the invention, the step of applying electrical pulses includes control the switch to output the electrical pulses to only those electrodes that have been selected based on a determination of which electrodes are near the stenosis.

In another aspect of the invention, the method further comprises determining at least one individualized electrical parameter for each pair of electrodes based on the determination of which electrodes are near the stenosis.

In another aspect of the invention, the electrical parameter includes Voltage or pulse duration.

In another aspect of the invention, the method further comprises determining at last one individualized electrical parameter for each pair of electrodes based on the depth and proximity of the stenosis in relation to the electrode positions.

In another aspect of the invention, the method includes determining an individualized voltage level to use for each pair of electrodes based on the depth of the restenosis near the each pair.

In another aspect of the invention, which electrodes are near the stenosis is determined by one or more imaging markers disposed near the electrodes.

In another aspect of the invention, the imaging markers include a radiopaque marker capable of rendering an image on any imaging modality, such as CT or IVUS.

In another aspect of the invention, which electrodes are near the stenosis is determined by applying test pulses to different pairs of the electrodes and measuring at least one electrical characteristic of the stenosis cells for the different pairs of electrodes.

In another aspect of the invention, the step of determining includes measuring an electrical resistance as the at least one electrical characteristic of tissue cells.

In another aspect of the invention, the method further comprises displaying a graphical representation and identification of the electrodes in positional relationship to the stenosis. In other words, electrode numbers are shown in relation to the position of the lesion so as to enable a user to determine which electrodes are the closest to the lesion and which electrodes close to the deepest part of the lesion.

In another aspect of the invention, the method further comprises displaying a graphical representation of the stenosis and a graphical representation and identification of the electrodes in positional relationship to the stenosis.

In another aspect of the invention, the method comprises electroporating tissue for the purpose of facilitating electrochemotherapy or electrogenetherapy, wherein cells are reversibly electroporated instead of killed, or the treatment is administered without necessarily killing cells or target tissue. Such methods can include inserting into a vessel an electrode having a plurality of elongated electrically conductive wires disposed lengthwise along the electrode and circumferentially spaced a selected distance from one another; orienting the electrode within the vessel to provide one or more of the electrically conductive wires in position to deliver one or more electrical pulse to target tissue; selecting one or more but less than all of the electrically conductive wires for administering the electrical pulse(s); administering the electrical pulse(s) from the selected electrically conductive wires to deliver the electrical pulse(s) to the target tissue and less than all vessel circumference; and wherein the administering is performed for a time and under circumstances sufficient to deliver drugs or genes to the target tissue or a portion thereof.

Such electrodes can also be used to enable directional targeting for other electroporation based therapies as well. For example, methods of directional targeting for selective macromolecule delivery, such as gene transfer are another application for electrodes of the invention. More particularly, the electrodes can be used for delivering insulin-making genes to pancreatic islets by way of the splenic artery; or can be used in chemotherapy treatments, especially for tumors; or can be used for other improved drug uptakes, such as for non-cancerous drug transports as well. Indeed, devices of the invention can be used as a device for directionally delivering any number of electrically-relevant interventional procedures to be delivered in a radially directed manner through blood vessels. Yet other applications include directional radiofrequency ablation or deep-brain stimulation, to name a couple.

According to another embodiment of the present invention, a medical device for treating a stenosis of a tubular body part by non-thermal irreversible electroporation is provided. The device includes a pulse generator, a balloon catheter, and at least three individually addressable (electrically independent) electrodes. The pulse generator generates electrical pulses in an amount which is sufficient to induce irreversible electroporation of cells of a stenosis to be treated, but which is insufficient to induce thermal damage to substantially all of the cells of the stenosis. The electrodes are positioned and spaced apart along the balloon, and electrically independent from each other. The electrodes are adapted to receive the electrical pulses from the pulse generator such that substantially all of the cells of the stenosis are killed by non-thermal irreversible electroporation. The ability to select which electrodes to energize based on the proximity of the lesion to the electrodes allows more precise targeting of the lesion while minimizing possible damage to surrounding healthy tissue.

In another aspect of the invention, the medical device further comprises a switch connected between a pulse generator and the electrodes, and adapted to connect the pulse generator output to any pair of the electrodes independent of the other electrodes.

In another aspect of the invention, the medical device further comprises a treatment control module adapted to control the switch to output the electrical pulses to those electrodes that have been selected based on a determination of which electrodes are near the stenosis.

In another aspect of the invention, the treatment control module is adapted to determine at least one individualized electrical parameter for each pair of electrodes based on a determination of which electrodes are near the stenosis.

In another aspect of the invention, the electrical parameter of the treatment control module includes Voltage or pulse duration.

In another aspect of the invention, the medical device further comprises one or more imaging markers disposed near the electrodes to determine which electrodes are near the stenosis.

In another aspect of the invention, the medical device comprises imaging markers that include a radiopaque marker.

In another aspect of the invention, the treatment control module is adapted to determine which electrodes are near the stenosis by applying test pulses to different pairs of the electrodes and measuring at least one electrical characteristic of the stenosis cells for the different pairs of electrodes.

In another aspect of the invention, the treatment control module is adapted to measure an electrical resistance as the at least one electrical characteristic of tissue cells.

In another aspect of the invention, the treatment control module is adapted to display a graphical representation and identification of the electrodes in positional relationship to the stenosis.

In another aspect of the invention, the treatment control module is adapted to display a graphical representation of the stenosis and a graphical representation and identification of the electrodes in positional relationship to the stenosis.

In another aspect of the invention, the treatment control module is adapted to determine at last one individualized electrical parameter for each pair of electrodes based on the depth and proximity of the stenosis in relation to the electrode positions.

In another aspect of the invention, the treatment control module is adapted to determine an individualized voltage level to use for each pair of electrodes based on the depth of the restenosis near the each pair.

In another embodiment of the invention, a method for treating a lesion of a tubular body part by non-thermal irreversible electroporation is provided. The method includes: (a) inserting into the tubular body part a plurality of elongated electrodes disposed lengthwise and circumferentially spaced a selected distance from one another; (b) positioning the electrodes within the tubular body part to provide one or more of the electrodes in position to deliver a plurality of electrical pulse to a target lesion; (c) selecting electrodes among the plurality of electrodes for administering the electrical pulses; and (d) administering the electrical pulses through only the selected electrodes to the target lesion in an amount which is sufficient to induce irreversible electroporation of cells of the target lesion, but which is insufficient to induce thermal damage to substantially all of the cells of the target lesion such that substantially all cells f the target lesion are killed by non-thermal irreversible electroporation.

In another aspect of the invention, the electrode comprises a flexible catheter and inflatable balloon and the electrodes are disposed lengthwise along and are circumferentially spaced around a surface of the inflatable balloon.

In another aspect of the invention, the method further comprises determining an orientation of the electrode within the tubular body part by imaging, wherein the electrode comprises at least one imaging marker for determining location of the electrodes.

In another aspect of the invention, the method further comprises measuring a distance between the imaging markers and using the distances to calculate rotational orientation of the electrode.

In another aspect of the invention, the one marker is radio-opaque.

In another aspect of the invention, at least two radio-opaque markers and at least one intravascular ultrasound marker are provided on or near an inflatable balloon.

In another aspect of the invention, the selection step includes: (a) administering one or more test pulses through any one or more of pairs of the electrodes; (b) determining from the test pulses one or more electrical characteristics of tissue subjected to the test pulses and based on the electrical characteristics further determining a depth of the target lesion; and (c) generating a protocol for administering higher voltage electrical pulses between electrode pairs positioned for treating deep restenosis and for administering lower voltage electrical pulses between electrode pairs positioned for treating shallow restenosis.

In another aspect of the invention, the test pulse or signal is a non-electroporating test pulse.

Additionally, in embodiments having an inflatable balloon, the electrically conductive wires (electrodes) can be disposed lengthwise along the electrode and can be circumferentially spaced around the electrode. In preferred embodiments, the electrically conductive wires are disposed circumferentially around the electrode and in contact with a surface of the inflatable balloon.

According to methods of the invention, orientation of the electrode within a body or vessel can be determined. This is particularly helpful for example in situations where it is desired to treat only a portion of the circumferential surface area of a blood vessel. In this situation, it may be desired to know the location of fewer than all of the electrically conductive wires relative to the location of asymmetrical restenosis within a blood vessel. Knowing the relative location of the wires, a practitioner can selectively energize only that portion of the electrode to treat the restenosis site thus leaving intact healthy tissue remaining on other portions of the circumferential surface area of the blood vessel. One such technique can employ an electrode comprising at least one imaging marker and determining the location of the electrically conductive wires by imaging the device in a body or vessel.

Using techniques of the invention and an electrode with at least one imaging marker, methods of the invention can include measuring a distance between the imaging markers and using the distances to calculate rotational orientation of the electrode.

Imaging markers disposed in the electrodes of the invention can be radio-opaque. In embodiments, electrodes can comprise at least one radio-opaque marker. In preferred embodiments, the electrodes can comprise at least two radio-opaque markers and at least one intravascular ultrasound marker. Further, there can be a plurality of radio-opaque markers, each associated with an individual electrically conductive wire of the electrode.

Another method for determining orientation relative to stenotic tissue of electrically conductive wires of an electrode can comprise: (a) inserting into a treatment area, such as a vessel, an electrode having a plurality of electrically conductive wires; (b) administering one or more test signals between two of the electrically conductive wires and subjecting tissue to the test signal(s); (c) determining from the test signal(s) one or more electrical characteristics of the tissue subjected to the test signal(s); and (d) comparing the electrical characteristic(s) to one or more threshold to confirm whether the tissue subjected to the test signal(s) is stenotic and whether the electrically conductive wires are in position to deliver an electrical charge to target stenotic tissue.

The electrical characteristics of the tissue can be determined, e.g., from resistance measurements, impedance measurements, and electrical impedance tomography.

Further embodiments of the invention include a method of mapping depth of stenotic tissue in real-time comprising: (a) inserting into a vessel an electrode having pairs of electrically conductive wires; (b) administering one or more test pulse between any one or more, or all, of the electrically conductive wire pairs; (c) determining from the test pulse(s) one or more electrical characteristics of tissue subjected to the test pulse(s) and based on the electrical characteristics further determining depth of stenosis; and (d) generating a protocol for administering higher voltage electrical pulse(s) between electrically conductive wire pairs positioned for treating deep stenosis and for administering lower voltage electrical pulse(s) between electrically conductive wire pairs positioned for treating shallow stenosis.

Methods disclosed in this specification can be used for treating, preventing, and/or reducing stenosis. Thus, methods according to embodiments of the invention may include identifying stenotic tissue as the target tissue.

An irreversible electroporation medical device is also encompassed within the scope of the present invention and can comprise: (a) an intravascular catheter type electrode having an inflatable balloon and a plurality of electrically conductive wires disposed lengthwise along the electrode/balloon/catheter; and (b) a plurality of imaging markers, each disposed relative to an electrically conductive wire, such that the markers, when subjected to imaging, reveal the identification of each wire and the distance between markers from which rotational orientation of the electrode within a body can be determined. In embodiments, the electrically conductive wires can be elongated and circumferentially spaced around the electrode a selected distance from one another. In other embodiments, the electrically conductive wires can be annular and longitudinally spaced along the length of the electrode a selected distance from one another.

Such methods can employ devices comprising imaging markers that are radio-opaque. Preferably, methods can comprise an electrode with at least two radio-opaque markers and at least one intravascular ultrasound marker to be used in determining orientation of the electrode relative to a treatment site.

Systems are also included within the scope of the invention. Such systems can include an intravascular IRE system comprising: (a) at least one intravascular catheter type electrode having an inflatable balloon and a plurality of electrically conductive wires disposed lengthwise along the electrode and circumferentially spaced a selected distance from one another; (b) an electrical pulse generator in operable communication with and for delivering electrical pulses to the plurality of electrically conductive wires; and (c) a control system in operable communication with the electrical pulse generator comprising a computer program embodied in a computer-readable storage medium, which program when executed, enables a computer to perform a method comprising: (i) determining orientation of the electrically conductive wires relative to target tissue; (ii) selecting one or more but less than all of the electrically conductive wires for administering the electrical pulse(s); and (iii) energizing one or more of the electrically conductive wires to deliver the electrical pulse(s) to the target tissue.

Objects of the invention include computer programs for running the IRE methods, systems, and devices described in this specification. Such computer programs can be embodied in a computer-readable storage medium, which when executed, enables a computer to perform a method comprising: (a) determining orientation relative to target tissue of at least one electrically conductive wire of an electrode; (b) selecting one or more but less than all of the electrically conductive wires for administering electrical pulse(s); and (c) energizing the selected wires to deliver the electrical pulse(s) to the target tissue.

### BRIEF DESCRIPTION OF THE DRAWINGS

The accompanying drawings illustrate certain aspects of some embodiments of the present invention, and should not be used to limit or define the invention. Together with the written description the drawings serve to explain certain principles of the invention.

FIGS. 1A-B are schematic diagrams showing a representative embodiment of a catheter type electrode device according to the present invention, where FIG. 1A shows all components clear to see underlying structure and where FIG. 1B shows the labeled components with opaque inner components.

FIG. 1C is a schematic diagram representing the different layers and dimensions used in various numerical simulations of IRE in the coronary artery, in which (described from the innermost region to the outermost region) a catheter with electrodes, blood, plaque, and smooth muscle were modeled.

FIG. 1D is a schematic diagram of an electrode of the invention comprising a catheter type device with embedded electrodes at a longitudinal separation distance of 5 mm.

FIG. 1E is a schematic diagram illustrating an electrode embodiment of the invention which is a catheter type device with embedded electrodes that can be used for IRE treatment of neointimal hyperplasia.

FIGS. 2A-B are schematic diagrams showing a cross-sectional view of a numerical model setup for symmetric restenosis (FIG. 2A) and asymmetric restenosis (FIG. 2B).

FIGS. 3A-D are schematic drawings illustrating a cross-sectional view of a representative numerical model output of electric field following a 100 μs pulse at 400 V, and more particularly: for treating symmetric restenosis with all 8 wires energized (FIG. 3A); for treating symmetric restenosis with 2 wires energized (FIG. 3B); for treating asymmetric restenosis with all 8 wires energized (FIG. 3C); and for treating asymmetric restenosis with 2 wires energized (FIG. 3D).

FIGS. 3E-F are schematic drawings illustrating a cross-sectional view of a representative numerical model output of temperature following a 100 μs pulse at 400 V, and more particularly: for treating symmetric restenosis with all 8 wires energized (FIG. 3E) and with only 2 wires energized (FIG. 3F).

FIG. 4 is a flowchart illustrating a method of selectively energizing one or more electrically conductive wires of an electrode for treating target tissue.

FIG. 5 is a schematic diagram of a representative electrical circuit for an electrode system of the invention, which circuit enables selective electrode energizing.

FIG. 6A is a schematic diagram of a balloon type catheter electrode comprising a plurality of electrically conductive wires disposed longitudinally over the length of the balloon.

FIG. 6B is a schematic diagram showing a cross-sectional view of the electrode illustrated in FIG. 6A comprising a radio-opaque marker disposed proximate each wire.

FIG. 6C is a schematic diagram illustrating markers disposed on a balloon type catheter electrode which would appear on an imaging apparatus, such as ultrasound, CT or X-ray, for identification/determination of the electrically conductive wires on the electrode.

FIGS. 6D-G are schematic diagrams illustrating orientation of the markers comprised in a balloon type electrode, which is shown at various orientations within a body.

FIG. 6H is a schematic diagram showing the cross section and a side view of a representative electrode with imaging markers to indicate electrode orientation.

FIGS. 6I-M are schematic diagrams showing a top view of the electrode in a blood vessel, illustrating how the imaging markers would show up on an imaging apparatus when oriented in various rotational orientations within the vessel.

FIG. 7 is a schematic diagram illustrating an IRE system of the invention.

FIG. 8 is a schematic diagram illustrating a control system for implementing methods of the invention and/or operating systems and devices of the invention.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Embodiments of the present invention include devices, systems, and methods employing expandable radially targeting electrodes. The electrodes can be designed for endovascular-based electroporation therapies and adapted for implementation with catheter-based guidance. This type of electrode with independently energized surfaces is also adaptable to any number of physical and clinical scenarios where radial targeting is desired.

Preferred electrode designs can be configured to use the combination of electrically conductive wires with an angioplasty balloon to bring the electrodes into direct contact with the targeted region, such as neointimal tissue. More specifically, designs of the present invention can employ for example eight independent conducting surfaces (wires or highly conducting solutions) to administer the electric pulses. The use of multiple conducting surfaces contained within the same electrode presents many advantages over the current state of the art designs. The electrically conductive surfaces of the electrode are not limited to wires. For example, any structure capable of providing a surface for delivering an electric charge can be used, including where the electrically conductive surfaces are bands or strips of material, or are printed on the surface of the balloon, or even further are compartments within an inflatable balloon comprising a highly conductive solution. Energizing the electrically conductive surfaces in sequence rather than collectively promotes an outward distribution of the electric field, reducing thermal effects and requiring lower voltages for treatment. Such techniques provide for an expanded treatment zone by energizing only a few of the electrically conductive wires at a time rather than the entire circumference of the electrode in one shot. In addition, by allowing independent spatial control of which portions of the electrode are energized (i.e., which wires), the electric field distribution may be customized in a manner that most effectively treats a specific lesion, such as an asymmetrical restenosis, while minimizing any damaging effects on healthy tissue. This allows a practitioner to more aggressively treat focally enlarged portions of a lesion while being more conservative on smaller regions of the same stenosis, which is important because it allows for improved accuracy in restenosis-ablation vascular-based surgeries. Further, applications include targeting other volumes located only on one side of a vessel, such as gene transfer targets just outside the vessel or tumor ablation using the tumor vasculature.

Accordingly, embodiments of this invention may be used as a catheter-style electrode to access target restenotic lesions from within the vessel. Embodiments of the present invention may also be used to target tissue disposed outside of the outer circumference of a vessel, such as an area between two or more blood vessels within a body. The use of a catheter electrode allows a practitioner to insert the electrode through a peripheral vessel, as is typical of current percutaneous vascular techniques, such as angioplasty and stenting. This renders treatments minimally invasive and advantageous over previous IRE investigations on blood vessels.

In the context of this specification, it should be noted that the terms “electrode,” “electrode device,” “device,” “balloon catheter electrode,” “catheter electrode,” or “device” are typically used to refer to the IRE medical device as a whole, which may include a catheter, balloon, and electrically conductive wires. The terms “conducting surfaces,” “electrically conductive wires,” “conducting wires,” “wire electrodes,” and “wires” are typically used to refer to a portion of the electrode, such as a pair or pairs of electrodes, which may be selectively energized to deliver electrical pulses. Notwithstanding these typical meanings, in some embodiments in this specification, the terms may be used interchangeably.

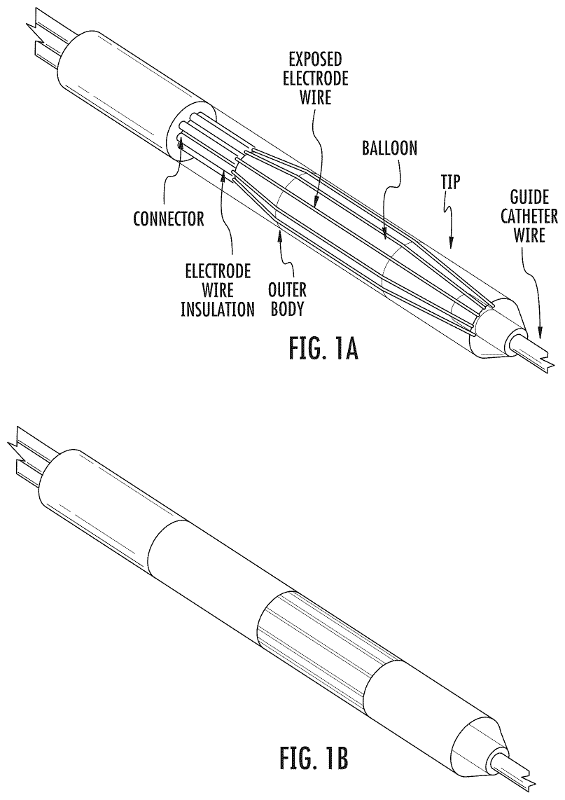

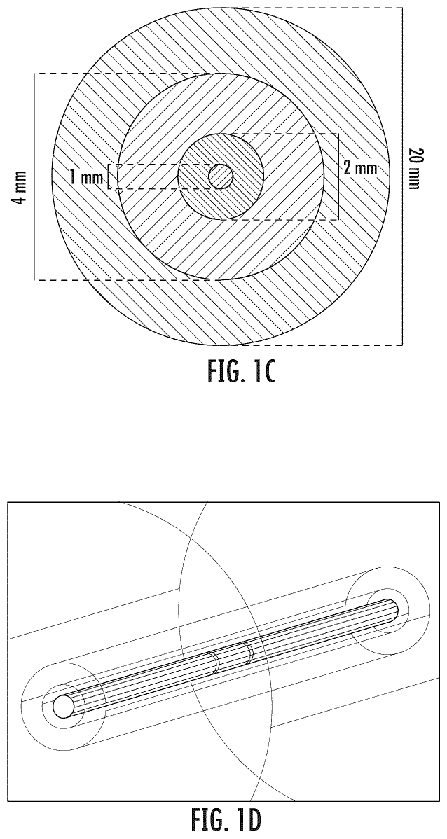

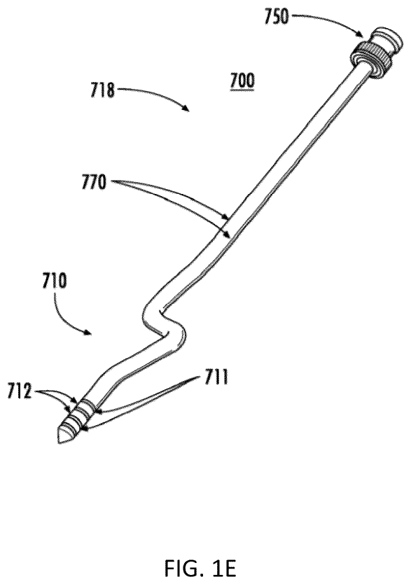

Exemplary electrodes according to embodiments of the invention are shown in FIGS. 1A-E. More particularly, as shown in FIGS. 1A-B, some of the components of the electrode device include a guidewire, tip, outer body, connector, balloon, and partially insulated conducting wires. The configuration depicted in this embodiment has a 2 mm diameter and is therefore designed for larger coronary vessels, such as the right coronary artery or left main artery, with proximal to medial lumen diameters of roughly 3.6 and 4.3 mm, respectively. See Dodge, J. T., Jr., B. G. Brown, E. L. Bolson, and H. T. Dodge, Lumen diameter of normal human coronary arteries, Influence of age, sex, anatomic variation, and left ventricular hypertrophy or dilation, Circulation, 1992, 86(1): p. 232-46. Such vessels may have typical restenotic lumen diameters of approximately 2.5 mm. See Radke, P. W., A. Kaiser, C. Frost, and U. Sigwart, Outcome after treatment of coronary in-stent restenosis—Results from a systematic review using meta-analysis techniques, European Heart Journal, 2003, 24(3): p. 266-273.

Modern micromachining techniques will allow for the construction of even smaller designs that may target smaller vessels, such as the distal portions and branches of the coronary arteries. Indeed, the devices of the present invention may also be scaled up for other applications. Accordingly, the diameter of the outer body of electrodes of the invention can range for example from about 0.1 mm up to about 5 cm. Preferably, electrode embodiments of the invention have an outer diameter ranging from about 0.5 mm to 5 mm, such as from about 1 mm to about 3 mm, such as about 1.5 mm to about 2 mm, or from about 0.75 mm to 3 cm.

In embodiments, the guidewire is the narrowest physical component and is used to direct the surgeon into the appropriate vessel. Although not typical, it can be hollow so that it may be used with a soluble contrast agent used for angiography and fluoroscopy, similar to typical endovascular therapies. See Schwartz, R. S., J. G. Murphy, W. D. Edwards, A. R. Camrud, R. E. Vliestra, and D. R. Holmes, Restenosis after balloon angioplasty, A practical proliferative model in porcine coronary arteries, Circulation, 1990, 82(6): p. 2190-200. The guidewire is usually of a smaller diameter than the diameter of the outer body of the balloon.

The guidewire or catheter forms the support for which all other components of the device are arranged. Over a portion of the catheter between the distal tip and the proximal end of the device is disposed an inflatable balloon. The balloon is secured to the catheter at the distal and proximal ends of the device. An inflation mechanism for providing a fluid into the area between the balloon and catheter is also provided. The balloon can be inflated during use with any inert fluid, such as saline, contrast fluid, air, or even low electrical conductivity sucrose solution. In embodiments where the electrically conductive wires are disposed on the inside of the balloon (between the balloon and the catheter) and where there is present a highly conductive fluid in the lumen (inside the balloon), the flow of the current would be preferentially through the fluid rather than through the wires which may result in a more diffuse electrical field. In preferred embodiments, a low-conductivity buffer is preferred as the fluid to inflate the balloon to more accurately treat target tissue.

The partially insulated conductive wires or electrodes provide for delivering the IRE electrical charge to target tissue from an electrical pulse generator. During use of the device, proximal ends of the wires/electrodes are in operable communication with a pulse generator. The wires can be hardwired directly to the pulse generator, or in preferred embodiments the electrode is equipped with a universal connector (or other connecting structure) for securing the electrically conductive wires/electrodes to the electrical pulse generator in an operably connective manner. For electrodes having a greater number of electrically conductive wires than the number of outputs available on a pulse generator it is desired to be used with, the electrodes and or pulse generator can be retrofitted or adapted accordingly to operably cooperate with one another. In embodiments, when there are more conductive wires on the electrode, e.g., 8 wires, than there are outputs on the pulse generator, e.g., 6 outputs, systems of the invention can comprise an electrode-generator interface that cooperates with the generator to switch which wires are active for a given pulse set in the overall sequence. More specifically, the interface can comprise a switch for switching between wires 4 and 8 for an 8-electrode system, since those wires would be least likely to be energized at the same time at 180° apart. Other examples for operation of an interface for treatments using basic 2-at-a-time pulsing pairs, the system can be connected to all 8 wires on the output, and just have a positive and negative input to take from the generator, where it would automatically switch pairs 1-2 to 2-3 and so on, while the generator's positive and negative outputs (port 1 and 2) would change pulse set 1 at (ex.) 2000 V (wires 1-2), then change to 1500 V (wires 2-3) for the second.

In this embodiment, the electrically conductive wires are elongated and are disposed along the length of the balloon. The wires are spaced a selected distance from one another around the circumference of the electrode. In FIGS. 1A-B, there are eight electrically conductive wires/electrodes circumferentially spaced around the balloon. The distal ends and proximal ends of the electrically conductive wires/electrodes are protected or encased by an outer insulative body, while a medial portion of the wires/electrodes is exposed to the atmosphere or vessel wall when implanted.

The distal tip of the balloon preferably includes a conical distal end to allow easy advancement through the vessel lumen. The conical distal end is preferably formed from or is in operable communication with a portion of the outer body of the device. The outer body of the device is an insulative encasing providing protection for the components of the device and for controlling the amount of exposure of the electrically conductive wires. Another portion of the outer body of the device provides for a proximal encasing disposed at the proximal end of the electrode. In embodiments, there is a gap or separation distance between the distal and proximal encasings or portions of the outer body. This gap or unprotected area of the electrode exposes the electrically conductive wires to the atmosphere.

A connector is disposed proximally of the balloon. The connector is hollow to allow movement with respect to the guidewire, and has an internal chamber that extends all the way back to the proximal ends of the balloon catheter that can carry fluids, such as physiologic saline or air or low electrical conductivity sucrose solution.

The balloon is attached to the connector. Running through the body/connector assembly is an array of 8 conducting 35 gauge wires (0.15 mm). These wires are insulated throughout the electrode apparatus until they reach the highly insulative balloon, after which they are exposed. In embodiments, the conducting wires can be attached to the proximal portion of the connector, but their distal ends are free and enclosed within the casing of the tip. This allows free expansion of the wires with inflation of the balloon. In other embodiments, the wires can be attached to the surface of the balloon and/or the distal ends of the wires attached to the distal tip, or distal outer body, or distal portion of the connector of the electrode. One example of an electrode balloon catheter is disclosed in U.S. patent application Ser. No. 12/413,332 filed Mar. 27, 2009 and entitled “Irreversible Electroporation Device And Method For Attenuating Neointimal”, which is incorporated herein by reference.

During use with the electrode inserted into a blood vessel, when the balloon is inflated with a fluid, the electrodes (electrically conductive wires) are expanded, and placed into contact with the targeted neointimal tissue disposed within the inner circumference of the blood vessel. In the embodiment shown, the length of exposure of each electrode is about 5 mm. However, larger exposure lengths may be used to treat diffuse restenosis lesions. See Rajagopal, V. and S. G. Rockson, Coronary restenosis: a review of mechanisms and management, The American Journal of Medicine, 2003, 115(7): p. 547-553. In specific embodiments, the exposure length of the electrically conductive wires and thus the exposed portion of the electrode can range from about 0.1 mm to about 5 cm, such as from about 0.5 mm to about 3 cm, or from about 0.8 mm to about 2 cm, or from about 1 mm to about 1 cm, or from about 1.5 mm to about 0.5 cm, or from about 2 mm to about 10 mm, or from about 2.5 mm to about 7 mm, or from about 3 mm to about 6 mm, such as from about 3.5 mm to about 4mm, etc.

FIGS. 1C-D provide another device embodiment of the invention. In this embodiment, the electrodes can comprise electrically conductive wires that are longitudinally spaced a selected distance from one another along the circumference of the balloon. As shown in FIG. 1C, an electrode of about 1 mm in diameter can be inserted lengthwise into a blood vessel. For illustration purposes, the blood vessel here has an outer diameter of about 20 mm and, when healthy, an inner diameter of about 4 mm. As illustrated in this case here, however, there is plaque build up, stenosis, or restenosis in the blood vessel, leaving only about a 2 mm inner diameter for the flow of blood through the vessel. As shown, an IRE device according to the invention is inserted into the blood vessel and used to ablate all or a portion of the growth within the vessel that is obstructing blood flow. In embodiments, there can be multiple rings (instead of 2) spaced along the longitudinal length of the electrode, and the applied voltages between each ring-pair (2 conductive regions energized) or ring-set (>2 conductive regions energized at a time). Preferably, the IRE device is oriented rotationally within the blood vessel to deliver one or more electrical charge(s) to target tissue disposed only around a portion of the inner circumference of the blood vessel. To further illustrate placement of the electrode within a blood vessel, FIG. 1D provides a perspective view of an electrode device embodiment of the invention inserted lengthwise into a blood vessel with the electrically conductive wires disposed proximate target tissue.

FIG. 1E provides a representative example of another electrode configuration according to embodiments of the invention. Examples of other configurations that can be used are disclosed in US Published Patent Application No. 2010/0030211, filed Jun. 24, 2009; US Published Patent Application No. 2001/0044596, filed May 4, 2001; and US Published Application No. 2009/0247933, filed Mar. 27, 2009.

The device 700 illustrated here in FIG. 1E, provides a minimally invasive microsurgical tool that uses IRE in coronary arteries to treat neointimal hyperplasia. Generally, the electrode 700 is a catheter type device with embedded active 712 and ground 711 electrically conductive wires. The electrically conductive wires 711, 712 are annular in shape and are disposed lengthwise along the length of the distal tip 710 of the electrode 700. The conductive wires 711, 712 are spaced a selected distance from one another longitudinally along the length of the electrode 700 and are separated by sections of insulation. The electrode 700 is compatible with existing electroporation electronics and comprises a universal connector 750 for connecting the proximal end 718 of the electrode 700 in operable communication with an electrical pulse generator. Existing systems that can be used and/or adapted for use with devices and methods of the invention include the NanoKnife® system from AngioDynamics® of Latham, NY. A portion of the electrically conductive wires 711, 712 is encased within the outer body 770 of electrode 700, or electrically conductive leads run from the electrically conductive wires 711, 712 along the length of the electrode 700 from the distal tip 710 to the proximal end 718 for operable communication with an electrical pulse generator.

The electrically conductive wires can comprise any type of conducting metal, such as platinum/iridium. Different materials will have different radio-opacity and can be selected according to this characteristic to achieve a particular result. For example, silver is much more radio-opaque than titanium and thus some embodiments of electrodes of the invention can have titanium conductive surfaces/wires, while using silver for the markers. To ensure biocompatibility, embodiments of the electrode can be sheathed with an insulating polyurethane jacket 770 to enclose the electrically conductive wires leading to the electrical pulse generator. In embodiments, the electrical conducting wires do not need to be entirely conducting. For example, the electrical conductive surfaces can comprise a portion or portions with an insulating coating (especially near their base). The exposed portions or surfaces of the electrically conductive wires 711, 712 on the distal tip 710 can be any thickness and width. Likewise, the amount of separation distance between the electrically conductive wires at the distal tip 710 can be any amount, and the electrodes can comprise any number of conductive wires. In embodiments, the electrodes can be configured in a manner to provide for an adjustable separation distance between electrically conductive wires, and/or an adjustable amount of exposed conductive surface.

This embodiment is constructed as a thin device, which allows for easy navigation through the cardiovascular system directly into the treatment site. In embodiments where there is no guidance catheter placed first (as in FIG. 1E), the electrode can comprise a J-shaped tip (or similar shape) as is common in angioplasty and catheter-based interventions so as to enable guidance of the electrode through the vasculature to reach the target site. Such an asymmetric tip could also be used as a source for determining rotational orientation for this particular embodiment. The electrically conductive wires, separated by an insulating material, generate the electric field distribution that determines the IRE treated regions. Representative dimensions of the electrode device, such as about 0.5 mm in diameter, ensures that it is feasible to be placed in the coronary artery since it is smaller than those already used in catheterization. The diameter or width is thus on the order of 0.5 mm to 1 cm. Preferably, the diameter or width is about 0.5 mm to about 5 mm, such as about 1 mm, 2 mm, 3 mm, or 4 mm. The length of the device is not particularly limited, but is generally set such that a surgeon can use the device comfortably to treat lesions at any position in the body. Thus, for human use, the device is typically on the order of 40 cm or less in length, such as about 30 cm, 25 cm, or 15 cm, whereas for veterinary use, the length can be much larger, depending on the size of animal to be treated. For treatment of human brain tumors, the length can be on the order of 40 cm.

The device can be customized by varying the diameters and separation distances of the electrically conductive wires, thus unique IRE treated areas can be predicted using mathematical models. As a result, successful treatment for neointimal hyperplasia is ensured due to the ability to match different plaque sizes and shapes.

Further, in some embodiments, the IRE device, or a portion thereof, is flexible. A flexible device is advantageous for use in accessing lesions non-invasively or minimally invasively through natural body cavities. In embodiments where the device or a portion of it is flexible, the shape of the device can change based on contact with body tissues, can be pre-set, or can be altered in real-time through use of wires or other control elements, as known in the art, for example in use with laparoscopic instruments.

Smooth muscle cells are the primary component of the neointimal hyperplasia typical of in-stent restenosis. See Rajagopal, V. and S. G. Rockson, Coronary restenosis: a review of mechanisms and management, The American Journal of Medicine, 2003, 115(7): p. 547-553. In order to kill these cells without damaging the healthy vessel architecture, it is desirable to harness the non-thermal mechanism of IRE to kill cells without inducing thermal damage. Mitigating thermal damage allows the extracellular matrix, nerves, and other sensitive structures to be spared. This allows for healthy regrowth of the tissue.

The primary factor determining the effect of an electroporation procedure is the electric field to which the tissue is exposed. However, IRE protocols have a variety of electrical pulse parameters that may also affect the toxicity of the treatment. In addition to the electric field, these include pulse shape, number of pulses, pulse length, and repetition rate. The thermal effects of an IRE treatment during a pulse are a direct function of the conductivity of the tissue and the voltage to which it is exposed. Therefore, minimizing the thermal effects for a particular tissue type may be done by finding the minimum required electric field, and thus applied voltage, to kill the cells in the tissue.

To this end, pulsing parameters and electrode configurations according to embodiments of the invention can include any combination of any of the following: a pulse length in the range of about 1 μs to 1 ms; a number of pulses ranging from 1 to 10,000; an electric field distribution for each conductive wire pair and/or across a treatment region ranging from about 5-5,000 V/cm; a total electrical charge delivered by way of each conductive wire pair and/or across a treatment region of about 0.1 to about 500 mC; a frequency of pulse application ranging from about 0.001-100 Hz; a frequency of pulse signal ranging from about 0-100 MHz; a pulse shape that is square, exponential decay, sawtooth, sinusoidal, or of alternating polarity although the currently favored pulse shape is a biphasic DC pulse; a positive, negative, and neutral electrical charge pulses (changing polarity within the pulse); a resulting current in the treated tissue ranging from about 0 to about 100 amps; from 1-20 electrodes and/or electrically conductive wires; an electrode and/or electrically conductive wire separation distance ranging from about 0.1 mm to about 5 cm; and multiple sets of pulse/electrode parameters for a single treatment, including changing any of the above parameters within the same treatment, such as removing the electrodes and replacing them in different locations within the tissue or changing the number of electrodes, to specialize/customize outcome.

For example, in embodiments a pulse length in the range of about 1 μs to 1 ms, such as from about 5 μs to about 0.5 ms, or from about 10 μs to about 0.1 ms, or from about 15 μs to about 95 μs. Pulse lengths of 20 μs, 25 μs, 30 μs, 35 μs, 40 μs, 45 μs, 50 μs, 55 μs, 60 μs, 65 μs, 70 μs, 75 μs, 80 μs, 85 μs, 90 μs, 110 μs, 150 μs, or 200 μs, and so on are also acceptable. The number of pulses can range for example from 5 to 5,000, or from about 10 to 2,000, or from about 20 to 1,000, or from about 30 to 500, or from about 50 to 200, or from about 75 to 150, or from about 90 to 120, or from about 95 to 110, or about 100 pulses.

Typically, the electric field distribution for each conductive wire pair and/or across a treatment region for IRE is performed using voltages ranging for example between 1500 V/cm to 4,000 V/cm. Voltages of much lower power can also be used, including using less than about 1500 V/cm. Applied fields of about 500 V/cm to 1000 V/cm can be used, or even of about 10 V/cm to about 750 V/cm, such as from about 50 V/cm to about 200 V/cm, or an electric field distribution of about 75 V/cm to about 100 V/cm. For example, in the treatment of brain tumors, typically, an applied field of less than 1000 V/cm can be used. Electrical pulse generators that can be used include those capable of delivering from 0 to about 5,000 V, such as the NanoKnife® system of AngioDynamics®, which for example can deliver from 0-3,000 V.

In preferred embodiments, a total electrical charge delivered by way of each conductive wire pair and/or across a treatment region of about 0.5 to about 25 mC can be used, such as about 1 mC to about 20 mC, or from about 1.5 mC to about 15 mC, or from about 2 mC to about 10 mC, or from about 5 mC to about 8 mC, and so on. Similarly, in preferred embodiments, the resulting current in the treated tissue can range for example from about 1 A to about 8 A, or from about 2 A to about 6 A, or from about 3 A to about 5 A, such as 4 A. Indeed, for certain applications the total electrical charge delivered can range from about 0.5 to about 500 mC, such as about 10 mC to about 200 mC, or from about 15 mC to about 150 mC, or from about 20 mC to about 100 mC, or from about 50 mC to about 80 mC, and so on. The resulting current in the treated tissue can range for example from about 1 A to about 80 A, or from about 20 A to about 60 A, or from about 30 A to about 50 A, such as 40 A. It is not uncommon for currents for IRE treatments to reach or exceed 40 and 50 amps, and it is further feasible to operate under even higher current with pulse generators capable of operating under such conditions as well. Currents are expected to be high in certain applications, especially when working in an area where the tissue or the medium is highly conductive, such as with blood present in a blood vessel. Pulse width, pulse shape, number of pulses, and the resultant current in the tissue can be adjusted to achieve specific target goals for limiting the total electric charge, and any of the specific values disclosed in this specification can be used to calculate the target expected charge.

Any number of electrically conductive wires or electrodes can also be used. However, in preferred embodiments 3 to about 18 electrodes are used, such as 3 to 16, or from about 3 to 15, or from 4 to 12, or from 5 to 10, or from 6 to 8. Any one or more of the electrodes/wires can be selectively energized to achieve a particular treatment result. Further, the separation distance between electrically conductive surfaces, such as electrically conductive wires and/or electrodes, can range from about 0.2 mm to about 4 mm, such as ranging from about 0.3 mm to about 3 mm, or from about 0.4 mm to about 2 mm, or from about 0.5 mm to about 1 mm, or from about 0.8 mm to about 4 cm, such as from about 0.9 mm to about 3 cm, or from about 1.2 cm to about 2 cm, or from about 1.5 cm to about 1.8 cm, and so on.

The electric field needed for a particular situation may be predicted through numerical modeling, allowing for reliable treatment planning. See Davalos, R. V., L. M. Mir, and B. Rubinsky, Tissue Ablation with Irreversible Electroporation, Ann Biomed Eng, 2005, 33(2): p. 223-231; Robert E. Neal II and R. V. Davalos, The Feasibility of Irreversible Electroporation for the Treatment of Breast Cancer and Other Heterogeneous Systems, Ann Biomed Eng, 2009, 37(12): p. 2615-2625; and Edd, J. F. and R. V. Davalos, Mathematical Modeling of Irreversible Electroporation for Treatment Planning, Technol Cancer Res Treat, 2007, 6(4): p. 275-286. To determine the efficacy of the electrode and understand the effects of the pulses on tissue, a numerical model has been developed capable of simulating treatments. This was done using a finite element software package, COMSOL Multiphysics (COMSOL, Stockholm, Sweden).

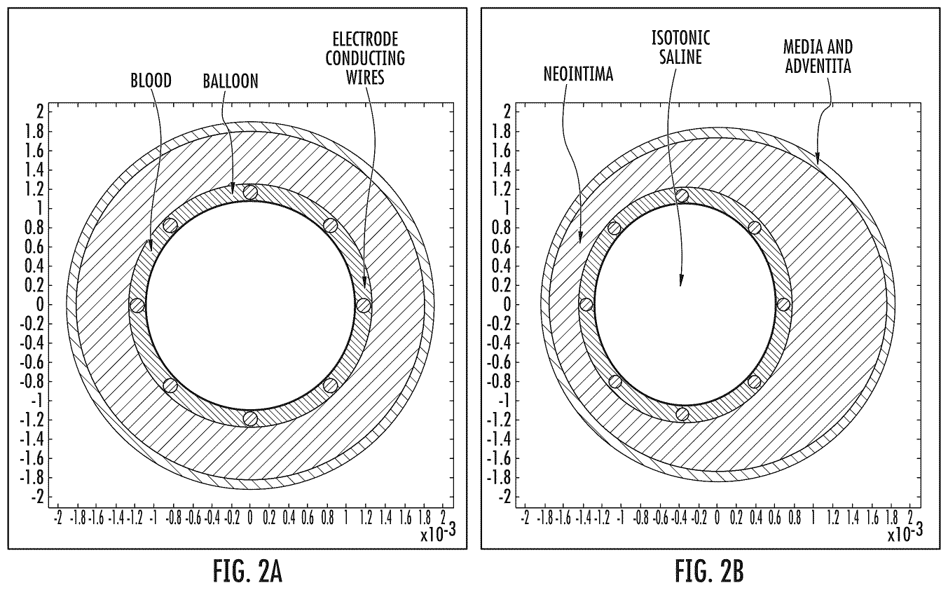

Two representative models were developed, simulating the cross section of a typical artery with a symmetric or an asymmetric restenosis. The model setups for the electrodes of FIGS. 1A-B are illustrated in FIGS. 2A-B.

In the numerical models illustrated, both use a blood vessel outer diameter of about 3.6 mm, with a combined tunica media and adventitia thickness of 200 μm, which are representative values derived from visual inspection of results from Maor, E., A. Ivorra, J. Leor, and B. Rubinsky, The Effect of Irreversible Electroporation on Blood Vessels, Technol Cancer Res Treat, 2007, 6(4): p. 307-312. These vessel layers are considered to be composed of collagen and elastin, as described in Saladin, K. S., The Circulatory System III-Blood Vessels, in Human Anatomy. 2008, Mcgraw-Hill: New York. p. 595-638.

The symmetric restenosis (FIG. 2A) assumed an equal amount of neointimal hyperplasia all around the vessel, reducing the luminal diameter to 2.5 mm, a cross-sectional reduction of 52% (stenosis of 48%). Inside the lumen, the model then contains a circular array of 8 electrode surfaces, each 0.15 mm in diameter, equally spaced around the neointimal tissue at 45° angles from the center, an angle switch of about 45 degrees. Inside the electrodes is a thin-walled balloon having an outer diameter of about 2.2 mm, modeled as rubber, and blood is assumed to be in the space between the balloon and the neointima. Inside the balloon is modeled as slightly hypotonic saline. The electrical and thermal properties of model components may be found in Table 1.

TABLE 1
Electrical and Thermal Properties Used in Numerical Modeling

| Property | Symbol | Tissue | Value | Units | Reference |
|---|---|---|---|---|---|
| Electrical | σ | Media and Adventitia | 0.25 | S/m | Carrara 2007 |
| Conductivity | | Neointima | 0.2 | | Carrara 2007 |
| | | Electrodes | 4.032 × 10⁶ | | Metals 1990 |
| | | Blood | 0.7 | | Carrara 2007; |
| | | | | | Duck 1990 |
| | | Balloon | 1 × 10⁻¹³ | | Serway 1998 |
| | | Isotonic Saline (0.15M) | 1.39 | | Gabriel 2009 |
| Density | ρ | Media and Adventitia | 1085 | kg/m³ | Werner 1988 |
| | | Neointima | 1085 | | Werner 1988 |
| | | Electrodes | 7850 | | Metals 1990 |
| | | Blood | 1059 | | Werner 1988 |
| | | Balloon | 2.17 | | |
| | | Isotonic Saline (0.15M) | 1000 | | Kenner 1977 |
| Thermal | k | Media and Adventitia | 0.55 | W/(m · K) | Werner 1988; |
| Conductivity | | | | | Bhattacharya 2003 |
| | | Neointima | 0.50 | | Werner 1988 |
| | | Electrodes | 44.5 | | Metals 1990 |
| | | Blood | 0.50 | | Duck 1990 |
| | | Balloon | 0.23 | | |
| | | Isotonic Saline (0.15M) | 0.50 | | |
| Specific | cₚ | Media and Adventitia | 3.20 | J/(kg · K) | Werner 1988 |
| Heat | | Neointima | 3.72 | | Duck 1990 |
| Capacity | | Electrodes | 475 | | Metals 1990 |
| | | Blood | 3.84 | | Duck 1990 |
| | | Balloon | 385 | | |
| | | Isotonic Saline (0.15M) | 3.84 | | |

See Carrara, N. Dielectric Properties of Body Tissues, Italian National Research Council: Institute for Applied Physics, 2007 (“Carrara 2007”) cited 2010, available from: http://niremf.ifac.cnr.it/tissprop/; see also Properties and Selection: Irons, Steels, and High-Performance Alloys, 10 ed. Metals Handbook, Vol. 1, 1990: ASM International (“Metals 1990”); see also Duck, F. A., Physical Properties of Tissue: A Comprehensive Reference Book, 1990, New York: Academic Press (“Duck 1990”); see also Serway, R. A., Principles of Physics, 2nd ed. Principles of Physics, 1998, Fort Worth, Texas; London: Saunders College Pub (“Serway 1998”); see also Gabriel, C., A. Peyman, and E. H. Grant, Electrical conductivity of tissue at frequencies below 1 MHz, Physics in Medicine and Biology, 2009, 54(16): p. 4863-4878 (“Gabriel 2009”); see also Werner, J. and M. Buse, Temperature profiles with respect to inhomogeneity and geometry of the human body, J Appl Physiol, 1988, 65(30): p. 1110-1118 (“Werner 1988”); see also Kenner, T., H. Leopold, and H. Hinghoferszalkay, Continuous High-Precision Measurement of Density of Flowing Blood, Pflugers Archiv-European Journal of Physiology, 1977, 370(1): p. 25-29 (“Kenner 1977”); see also Bhattacharya, A. and R. L. Mahajan, Temperature dependence of thermal conductivity of biological tissues, Physiological Measurement, 2003, 24(3): p. 769-783 (“Bhattacharya 2003”).

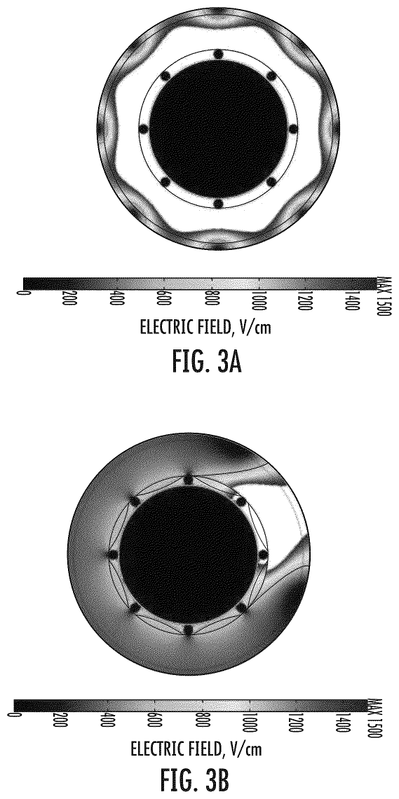

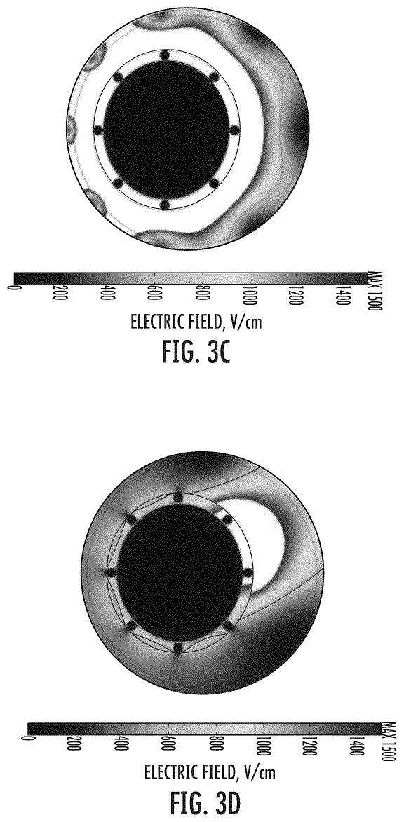

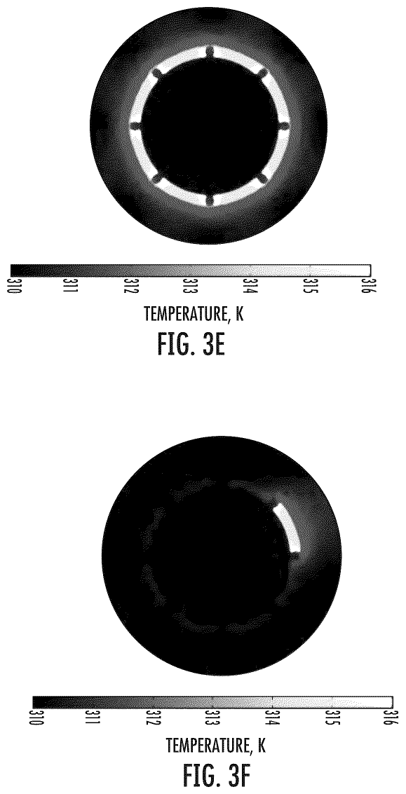

The representative numerical model was solved for the electric field distribution for a voltage of 400 V/cm. This was done either with all conducting surfaces energized in an alternating (V0-0-V0) fashion around the electrode, or with only two adjacent surfaces energized (one as V0 and one as ground). According to embodiments of the invention, any range of energized wire arrangements are possible (E1 at V0 and E3 at 0; or even E1 at V0, E4 at V0/3, and E7 at 0). Indeed, multiple electrodes can be energized while multiple others are set to ground. There is also no limitation on which electrodes can be energized and which are set to ground, which will depend on a particular treatment protocol being administered. FIGS. 3A-D show the resulting electric field distribution between 0 and 1500 V/cm, with a black contour at 637 V/cm, a typical IRE threshold taken from the literature. See Miklavcic, D., D. Semrov, H. Mekid, and L. M. Mir, A validated model of in vivo electric field distribution in tissues for electrochemotherapy and for DNA electrotransfer for gene therapy, Biochimica et Biophysica Acta, 2000, 1523: p. 73-83.

The representative numerical model output of electric field (FIGS. 3A-D) and temperature (FIGS. 3E-F) is illustrated following a 100 μs pulse at 400 V. More particularly, FIG. 3A illustrates treatment of symmetric restenosis with all 8 wires energized, while FIG. 3B illustrates treatment of symmetric restenosis with only 2 wires energized. Likewise, treatment of asymmetric restenosis with all eight wires energized is shown in FIG. 3C, while treatment of asymmetric restenosis with only two wires energized is shown in FIG. 3D. The black contour line shown in FIGS. A-D is 637 V/cm.

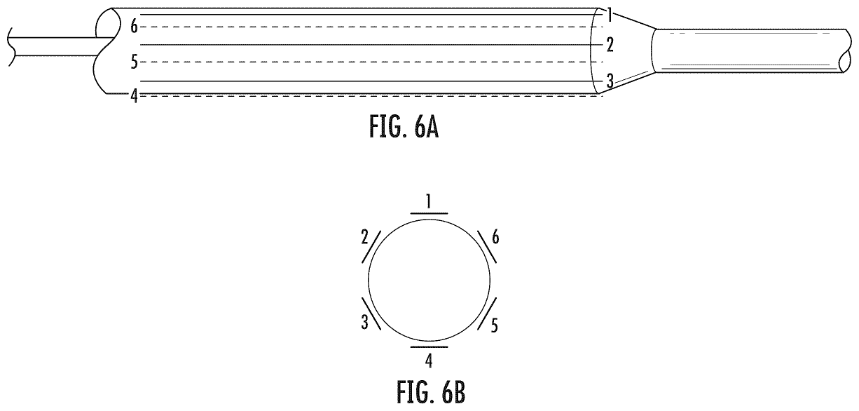

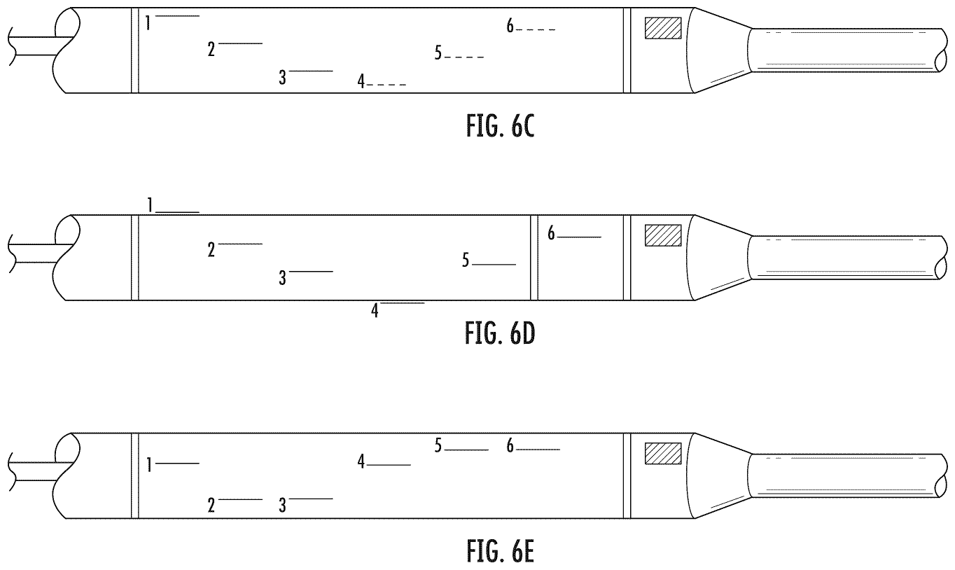

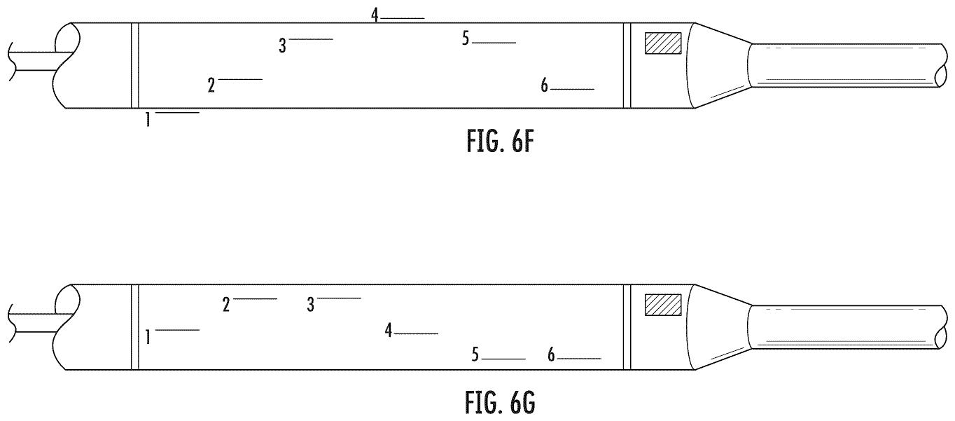

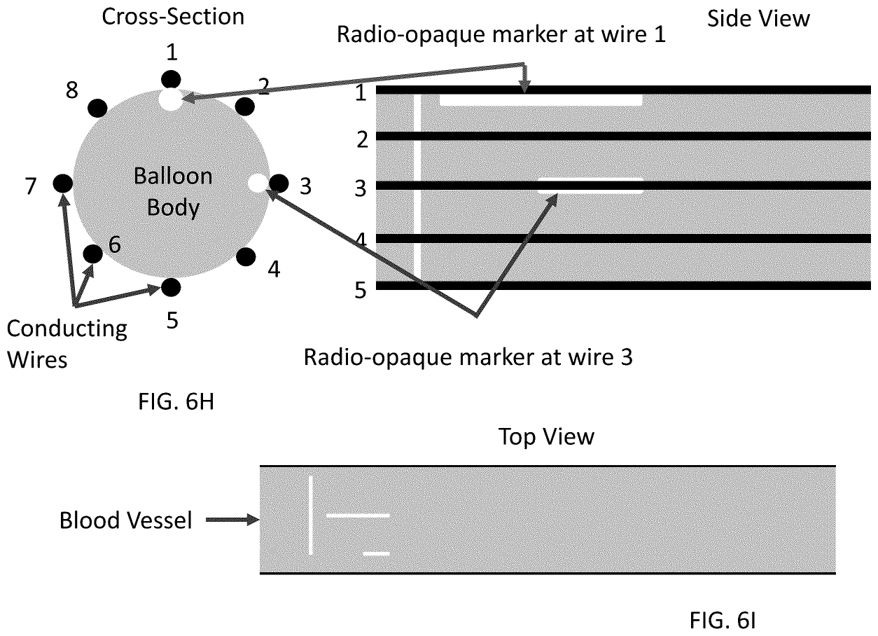

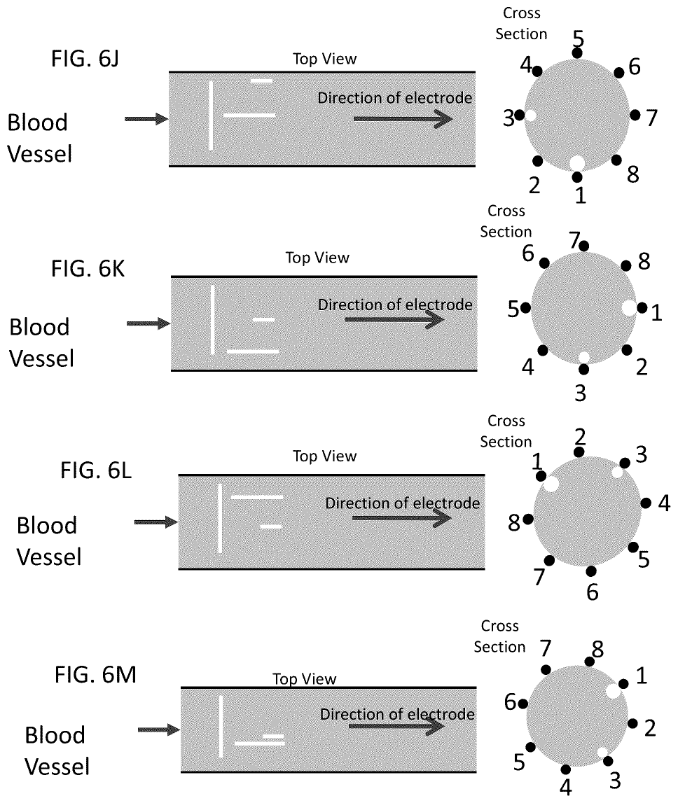

It is important to note that the devices of the invention can comprise any number of electrically conductive wires disposed in any manner on the electrode. In methods of the invention, any number of electrically conductive wires can be energized in any order and in any combination. For example, for an electrode having ten electrically conductive wires disposed around the circumference of the electrode and spaced circumferentially or longitudinally from one another and progressing upwardly in number order from 1 to 10, the wires can be selectively energized using all, or less than all of the wires around the circumference of the electrode or along the length of the electrode. Referring to FIG. 6A-6B, in such embodiments, wires 5 and 6 can be energized, then wires 2 and 4 energized, then wires 3 and 5 energized to treat a target region disposed proximate the area near electrodes 2-6. This leaves the area or substantially most of the area proximate wires 6 to 1 untreated.

Referring back to FIGS. 3A-3F, when all the wires of the 8-wire electrode were energized, it was found that only the corners of the outermost regions of the neointimal hyperplasia were not treated with 637 V/cm in the symmetrical stenosis; while there is a large gap at the expanded side in the asymmetrical case. In order to effectively treat the entirety of the asymmetrical stenosis, significantly larger voltages should be used. Since previous designs would have to energize all the conducting surfaces equally, the region of tissue exposed to IRE would likely expand beyond the vessel, possibly affecting healthy tissues, which highlights insufficiencies of previous catheter-based electrode designs regarding non-cylindrical stenoses.

Notably, when two adjacent wires were energized, the treatment margins of IRE (presumed at 637 V/cm) extend easily through the local neointimal tissue. This shows that lower voltages may be used if pairs of electrodes are energized in succession rather than simultaneously. Furthermore, treating in this way allows for a practitioner to finely tune the applied voltage for each sequence, locally extending treatment regions at thicker regions of restenosis while decreasing treatment margins at thinner regions. From the determination that a lower voltage is required when only two electrodes are energized at a time, it can clearly be seen how the proposed model may be used to investigate the effects of various treatment parameters in order to optimize treatments to be used in clinical and pre-clinical settings.

The electric potentials used to generate the electric fields used in IRE also cause Joule heating of the tissue. This is a function of the electric potential to which a bulk of tissue is exposed, its electrical conductivity, and the time for which it is exposed. The thermal effects of catheter type IRE are illustrated in FIGS. 3E-F. Representative numerical models for temperature output are illustrated for treatment of symmetric restenosis using all eight wires energized (FIG. 3E) and using only two wires energized (FIG. 3F). In order to accomplish complete IRE ablation of a targeted region without damaging the extracellular matrix and other sensitive structures, a comprehensive quantitative understanding of the thermal effects from a treatment protocol is vital. By numerically modeling these effects, one is able to determine the potential for any thermal damage to the tissue structures and adjust treatment plan protocols prior to application in order to minimize or eliminate this undesired form of potential damage. Therefore quantitative modeling of the thermal effects will be done through utilization of the numerical model previously described for understanding electric field behavior.

More particularly, the thermal behavior of tissue may be assessed using a modified Pennes Bioheat equation with the addition of a Joule heating term as outlined below:

$$∇ ( k ∇ T ) + {w}_{b} {c}_{b} ( {T}_{a} - T ) + {q}^{′′′} + σ {\vert ∇ Φ \vert}^{2} = ρ {c}_{p} \frac{dT}{dt} \qquad ( 1 )$$

where k is the thermal conductivity of the tissue, T is the temperature, c_{b} and cₚ are blood and tissue heat capacity, respectively, w_{b} is blood perfusion, Tₐ is arterial temperature, ρ is tissue density, σ|∇Φ|² is the joule heating term, and q″′ is metabolic heat creation. The outer vessel boundary was treated as adiabatic. See Davalos, R. V. and B. Rubinsky, Temperature considerations during irreversible electroporation, International Journal of Heat and Mass Transfer, 2008, 51(23-24): p. 5617-5622. Because the time scale of the electroporation pulses (microseconds) is much lower than those involved in metabolic heat generation and blood flow (see Werner, J. and M. Buse, Temperature profiles with respect to inhomogeneity and geometry of the human body, J Appl Physiol, 1988, 65(30): p. 1110-1118; and Gautherie, M., Y. Quenneville, and C. M. Gros, Metabolic heat production, growth rate, and prognosis of early breast carcinomas, Biomedicine, 1975, 22: p. 328-336), one is able to see that the dominant terms affecting change in temperature for a volume of tissue is the contributions of heat conduction from neighboring tissues and electroporation pulse induced Joule heating.

By eliminating the blood perfusion and metabolic heat generation terms and rearranging the terms, the equation becomes:

$$\frac{dT}{dt} = \frac{∇ ( k ∇ T ) + σ {\vert ∇ Φ \vert}^{2}}{ρ {c}_{p}} \qquad ( 2 )$$

For a single pulse of infinitely small duration, δt, the change in temperature may be described by:

$$dT = \frac{∇ ( k ∇ T ) + σ {\vert ∇ Φ \vert}^{2}}{ρ {c}_{p}} dt \qquad ( 3 )$$

From the modified Pennes' Bioheat equation above, it becomes evident that the controllable terms affecting electroporation-induced temperature changes are the magnitude of the electric field and the duration of the pulse. Assessment of thermal effects from the model would allow one to adjust the protocols to prevent thermal damage and understand its impact on the electric field distribution. For instance, the pulse length could be shortened, a low-conductivity gel could be injected into the tissue, or an actively-cooled electrode could be used to cool the tissue prior to and during pulsing.

The representative numerical model of FIGS. 3A-F has been evaluated for the temperature distribution resulting from a single 100 μs pulse. The initial condition for the temperature of the entire tissue was taken to be 310.15 K (37° C.), which is the physiological temperature. FIGS. 3E-F show the results when a voltage of 400 V was applied to the symmetric stenosis for the case of eight and two energized surfaces. From this, it can be seen that only a very small portion of blood near the electrodes reaches temperatures above 314 K, a change of 4 K. In addition, most of the thermal effects occur in the blood between the energized surfaces, with very little noticeable effect to the neointimal tissue.

To evaluate potential thermal damage, the maximum temperature could be compared to a typical threshold of protein denaturation and scarring of 50° C. (323 K). See Diller, K. R., Advances in Heat Transfer, in Bioengineering Heat Transfer, Y. I. Choi, Editor, 1992, Academic Press: Boston. p. 157-357. Because 316 K falls well below this temperature threshold, it is clear that the treatment protocols used in the first part of this numerically modeled example are able to fully treat the targeted region without inflicting thermal damage. It should be noted that thermal damage may occur at temperatures below 50° C. when carried out over a long period of time (such as hours, for example), and that the combined effects of many pulses may further increase the temperatures. However, for the current example of the numerical model outlined above, it may be assumed that the high perfusion rate of blood in an artery will rapidly dissipate the heat generated. In embodiments, however, where the balloon is inflated blood flow within the blood vessel may be greatly reduced or completely blocked and heat dissipation may occur in some other manner. This, in addition to the relatively long span between pulses (for example, meaning 0.25 seconds as compared to 0.0001 seconds of a 100 μs long pulse); allows one to expect that the temperature will return to physiologic temperatures prior to the next pulse, preventing it from ever exceeding the 50° C. threshold.

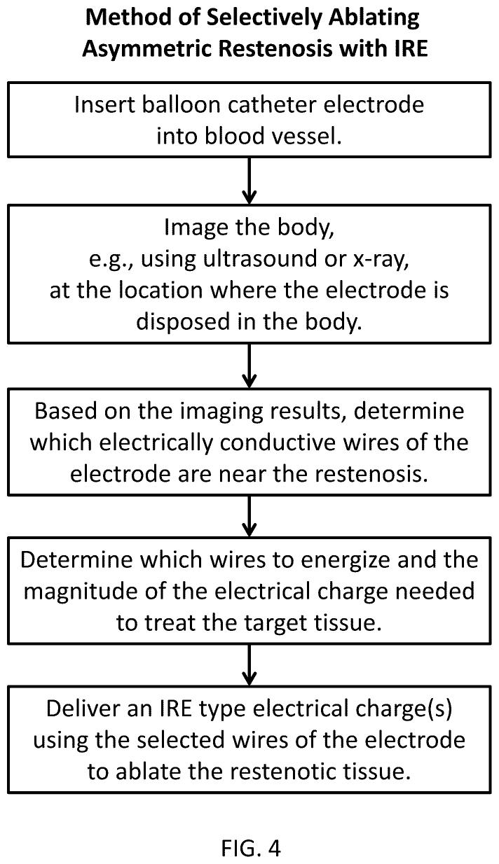

A method of selectively ablating asymmetric restenosis is illustrated in FIG. 4. Such methods can involve one or more of the following: inserting a balloon catheter electrode in a blood vessel; imaging the body in which the catheter is placed, for example, using ultrasound, x-ray, CT, MRI, etc.; based on the imaging results, determining which electrically conductive wires of the electrode are near the restenosis; determining which wires of the electrode to energize and the magnitude of the electrical charge needed to treat the target tissue; and delivering an IRE type electrical charge(s) between the selected wires of the electrode to ablate the restenotic tissue. These method steps can be used singularly or one or more together with other methods and/or method steps described in this specification. One of skill in the art will know how to modify the methods according to a particular result to achieve.

Methods of the invention can also include the capability of being able to synchronize the electrical pulses with the cardiac rhythm of the patient to avoid arrhythmia. This is especially important for treatments administered on coronary arteries, e.g., directly at the heart, where chances of arrhythmia are highest. In addition, treatment of arrhythmogenic regions of the heart from the inside is yet another application for the asymmetric ablation protocols of the present invention. In some situations, it is feasible that such treatments could be used in lieu of open heart surgery.

Preferred methods of embodiments of the invention are directed to electrically ablating tissue, with the method comprising: inserting into a vessel an electrode having a plurality of electrically conductive wires disposed lengthwise along the electrode and circumferentially spaced a selected distance from one another; orienting the electrode within the vessel to provide one or more of the electrically conductive wires in position to deliver one or more electrical pulse to target tissue; selecting one or more but less than all of the electrically conductive wires for administering the electrical pulse(s); administering the electrical pulse(s) from the selected electrically conductive wires to deliver the electrical pulse(s) to the target tissue and less than all vessel circumference; and wherein the administering is performed for a time and under circumstances sufficient to cause irreversible electroporation of the target tissue or a portion thereof.

Methods of the invention can also be used for reversible electroporation of tissue to assist or enable electrochemotherapies and/or electrogenetherapies. Even further, aspects of methods of the invention include inserting the electrode device into any organ or vessel which is not in particular a blood vessel, such as within the lymphatic system for treating undesired tissue such as lymphoma. Even further, the electrodes can be used in arrhythmogenic regions of the heart or tumor nodules in the lungs, which can be accessed through vessels of the respiratory tract such as bronchial tubes or blood vessels.

Methods of the invention can employ an electrode comprising a flexible catheter and inflatable balloon with the electrically conductive wires disposed lengthwise along and circumferentially spaced around the electrode, such as on a surface of the inflatable balloon.

In embodiments, the electrically conductive wires can be selectively energized, especially in a sequential manner across only a portion of the circumference of a blood vessel or other treatment area. For example, in embodiments where an electrode comprises eight electrically conductive wires, the method can comprise orienting only a portion of the wires proximate a target treatment area, such as wires 1, 2, and 3 of the eight-wire system. A selected number of pulses at a selected electrical charge can be administered between wires 1 and 2, then a selected number of pulses at a selected electrical charge (which may be different or the same as that applied between wires 1 and 2) may be delivered between wires 2 and 3. Then this pattern or a different pulsing protocol can be administered selectively and sequentially using selected wire pairs. In preferred embodiments, less than all of the wire pairs are used and less than all of the circumference of the electrode is energized during a treatment. In this manner, less than all of the surface area of a blood vessel can be subjected to the IRE.

More particularly, for example, 10 pulses of 50 μs in length at 500 V/cm can be delivered between a first selected electrically conductive wire pair, then 100 pulses of 100 μs in length at 2500 V/cm can be delivered using a second wire pair, then 50 pulses at 75 μs in length at 1000 V/cm can be administered using a third wire pair. This sequence of pulsing can then be repeated any number of times until a desired treatment outcome is reached. Alternatively, any one or more of the pulsing parameters can be changed during the treatment to modify the effect the pulsing protocol is having on the tissue. For example, a second round of pulsing using the first, second, and third wire pairs can be administered by changing the parameters for the third wire pair, such as by delivering 20 pulses that are 90 μs in length at 1500 V/cm. This round of pulsing protocols, or combinations of the protocols, can then be continuously and sequentially administered until a desired treatment result is achieved. By energizing only a portion of the circumference of the electrode (only the first, second, and third wire pairs), only a portion of a selected region of the body that surrounds the electrode, such as a portion of a blood vessel, is subjected to the IRE thus rendering the non-targeted regions unaffected. Changing parameters impacts the depth of IRE ablation. Accordingly, the treatment can be customized to ensure complete treatment of thicker stenotic segments without over-treating regions with shallower stenotic segments.

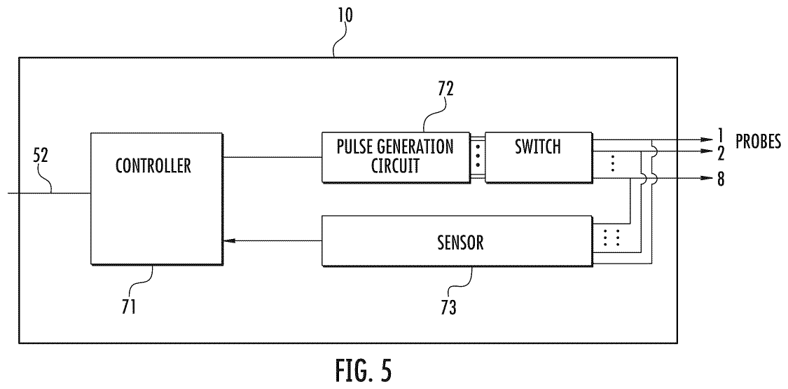

FIG. 5 is a schematic diagram illustrating a representative electrical circuit for an electroporation system according to embodiments of the invention. More particularly, FIG. 5 provides a schematic of an electrical circuit for an electroporation system, the system comprising a plurality of electrically conductive wires (electrodes) or solutions with high electrical conductivity (blood); a pulse generator and sensor(s) in operable communication with the probes; and a controller or control system in operable communication with the pulse generator and sensor(s). The controller in operable communication with the other components of the system together provide for a system capable of selective electrode energizing. The electrical circuit 10, in particular, comprises an electrical connection with a power source for delivery of electrical energy to the controller 71. The controller 71, alone or in combination with sensor(s) 73, in turns provides power to the pulse generation circuit 72. The pulse generation circuit 72 is in operable communication with a switch for delivering the electrical energy to one or more, all, or less than all of the probes. The switch is operably configured to selectively deliver electrical energy to the probes in any manner. In preferred embodiments, the switch is capable of providing electrical energy sequentially to each of the probes over the entire circumference of the electrode, or over only a portion of the circumference of the electrode. Likewise, the switch is capable of providing electrical energy to a single probe, or more than one probe, or combinations of any two probes, in combination with any pulse protocol using any number, or length, or intensity of electrical pulses. The switch and pulse generator are operably connected with any number of probes. Here, up to eight probes or electrically conductive wires are illustrated for this representative system.

As shown in FIGS. 6A-G, other aspects of embodiments of the invention include devices and methods for determining the identification, location and/or orientation of the electrode and/or electrically conductive wires when inserted into the body, and especially with respect to the location of target tissue, including asymmetrical stenosis in a blood vessel.

A representative embodiment is provided in FIGS. 6A-G. By equipping devices and systems of the invention with one or more imaging markers, the overall rotational orientation of the electrode as disposed in a body or vessel can be determined. As shown in FIG. 6A, an angioplasty balloon type catheter electrode can comprise six electrically conductive wires disposed longitudinally over the length of the electrode and circumferentially spaced a selected distance from one another around the circumference of the catheter or electrode. FIG. 6B, shows brief radio-opaque plugs (such as silver) provided on or in connection with one or more or all of the electrically conductive wires of the electrode. Here, an imaging marker is associated with each of the electrically conductive wires and is disposed in the electrode in a manner to provide the plugs progressing clockwise/counterclockwise around the wires of the electrode. The schematic of FIG. 6B provides a cross-sectional view of the device illustrating placement of the imaging markers in connection with the electrically conductive wires.

Although a 6-wire system is provided in FIGS. 6A-G, the approach could be used with any number of wires by altering the angle switch from each. In the context of this specification, the term “angle switch” is meant to refer to the angular distance of separation between electrically conductive wires around the circumference of the electrode. For example, an electrode with four wires would have an angle switch of about 90 degrees between wires, while an electrode with six wires has an angle switch of about 60 degrees. In preferred embodiments, electrodes of the invention comprise any number of electrically conductive wires ranging from 1 to 20, such as from 2-10, or from 4-8, or even from 5-6 wires. The wires can also be disposed in any orientation relative to the electrode, such as circumferentially and longitudinally spaced a selected distance from one another; or disposed longitudinally and spaced circumferentially a selected distance from one another; or the electrically conductive wires can be disposed in a spiral or helical manner around the circumference of the electrode.

Additionally, or alternatively, proximal and distal imaging markers can be provided at one or both ends of the electrode device. In preferred embodiments, the proximal and distal markers can comprise a radio-opaque material with an overall annular shape for disposing each imaging marker around the circumference of the electrode/catheter (to give a definitive start and finish).

With the electrode device inserted into the body of a patient, the region of the body where the device is disposed can be imaged, for example, using x-ray, ultrasound, MRI, CT, or angiography, for example. Depending on the shapes that show up on angiography/x-ray, the orientation of the electrode within the body can be determined, especially its rotational orientation within a blood vessel and relative to a stenotic region. In embodiments, any number of imaging markers can be used, such as from 1-25 and any number in between. In preferred embodiments, at least two markers are used, such as one marker to denote each electrically conductive wire. By measuring the 2D distance from one imaging marker to the next as revealed on an imaging modality, the rotation of the electrode can be determined.

Further, in embodiments, a differential echogenicity (extra bright/dark in ultrasound) can be placed at the distal tip and proximal portions at one or more specific electrically conductive wires so that the marker could be picked up on intravascular ultrasound (IVUS). Using these approaches, it is relatively easy to see wire orientations relative to any asymmetrical stenoses or other targets.

FIG. 6C illustrates an electrode embodiment of the invention comprising six electrically conductive wires, each with an associated imaging marker disposed on, proximal to, or in connection therewith. In this embodiment, the imaging markers for the electrically conductive wires are radio-opaque plugs disposed in a counterclockwise progressive manner, which are used to denote the location of each wire. Proximal and distal end imaging markers are also included, which show where the wire imaging markers begin and end. Optionally included is a hyperechoic slug disposed in association with wire 1. As illustrated, represented is the expected x-ray image of the balloon catheter electrode of FIG. 6A inserted in a body and disposed within the vessel in a plane parallel to the drawing sheet.

FIGS. 6D-G provide representative x-ray images of the electrode disposed at a rotational orientation relative to that shown in FIG. 6C. More particularly, as shown in FIG. 6D, the image illustrates the balloon catheter disposed in a body in same orientation as shown in FIGS. 6A and 6C. The representative x-ray image of FIG. 6E, indicates the balloon catheter electrode is oriented in the body with wire 1 oriented up toward and closest to the imaging device. Further, the orientation of the device of FIG. 6E is rotated upward out of the plane of the drawing sheet and 90 degrees relative to the position shown in FIGS. 6A, 6C, and 6D. If the device were then rotated another 90 degrees upward from the position shown in FIG. 6E, the imaging markers would be arranged as illustrated in FIG. 6F. Similarly, FIG. 6G shows rotation of the device another 90 degrees upward from the position shown in FIG. 6F, with marker 1 farthest from the imaging device, thus indicating wire 1 is 180 degrees rotated from the source of the imaging device.

Another embodiment can comprise a radio-opaque spiral rotating in a certain direction from a specific wire. The direction where it rotates from and ends on would give an exact orientation of the catheter.

In yet other embodiments, it is possible to use the wires themselves to identify which are the closest to the restenosis site for purposes of determining which electrodes to energize to treat a targeted area within the vessel. For example, using an electrical charge, a non-electroporating test signal (AC or DC pulse or pulses) can be injected between a pair of wires. Then, the electrical characteristics of the tissue lying between the electrode pair can be measured. The electrical characteristics measured can include, for example, resistance, impedance (complex impedance which includes real and imaginary parts), electrical impedance tomography, and so forth. The measured characteristics of healthy tissue will be different from that of restenotic tissue. The measured characteristics can thus be compared with threshold electrical characteristics determined by experiment, such as shown in U.S. Pat. No. 7,742,795, issued Jun. 22, 2010 to Minnow Medical Inc., incorporated herein by reference.

FIGS. 6H-M illustrate another of many potential ways to determine exact rotational orientation of the catheter electrode to ensure that differential targeting occurs in the desired region of the blood vessel. This example provides an 8-conducting wire electrode with a simple system of 2 radiopaque markers oriented 90 degrees apart (wires 1 and 3 ). The system would be appropriate for use with angiography, and the markers could be placed on the balloon below the electrodes, using a more radiopaque marker than the conducting wires themselves, such as silver. Including a circumferential marker in the device ensures starting at the correct wire (more useful for applications such as if every wire were to have a marker).

As shown, the imaging markers in this embodiment are different sizes to identify each marker and differentiate between the two. Here, wire 1′s marker is long and wire 3′s marker is short. This configuration allows the markers to overlap in length so the distance between the two can be easily measured to get an exact angle of rotation. A schematic providing a cross-sectional view and a side view of the device as it would appear as an image on an imaging device is shown in FIG. 6H. It is noted that in FIGS. 6H-M that the illustrations are not intended to represent exactly how the images would actually look on for example angiography (because more radiopaque materials show up darker), rather the schematics are intended to show how the position of the markers would identify the orientation of the device.

FIG. 6I illustrates how the electrode would look on angiography oriented with conducting wire 1 on top (assuming balloon and wires invisible to scanner); FIG. 6J illustrates the electrode oriented with conducting wire 1 on bottom; FIG. 6K illustrates electrode orientation with conducting wire 1 on right (relative to electrode direction); FIG. 6L illustrates the electrode orientation with conducting wire 1 at 10:30 orientation (relative to electrode direction); and FIG. 6M illustrates how the would look on angiography oriented with conducting wire 1 at 2:30 orientation (relative to electrode direction)—distance measured between the two markers will give the amount of angle electrode.

Determining the proximity of each wire to the restenotic region is possible due to the differences in impedance between restenotic tissue (densely packed disorganized cells) and blood vessel walls (endothelial layer surrounded by connective tissue). In embodiments, a sequence of non-electroporating electrical test pulses (AC or DC) between any one or more, or all, conducting wire pairs around the perimeter of the catheter could be used to determine the extent/depth of restenosis between each pair in real-time while the balloon is inside the tubular body part. This data could then be used to generate a “map” of restenosis depth around the electrode. This data can further be used to generate a protocol for how strong the electrical pulses should be between each wire pair to ablate all of the restenosis for that portion of the vessel. In other words, greater restenosis depth between electrode pairs would have a greater change in properties, which would guide the practitioner to use higher voltages for that pair to ensure ablation of the entire depth, while areas without as much depth would warrant electrical pulses of lower voltage(s). Accordingly, a machine/program could be used to automatically customize pulse parameters for each pair based on restenosis geometry.

In one embodiment, the treatment control module 54 has been programmed to display on the display device 11 a graphical representation of the stenosis and a graphical representation and identification of the electrodes (e.g., electrode numbers) in positional relationship to the stenosis. Graphically, the image would be similar to that shown in FIG. 2B, except the electrodes would be numbered such that a user would be able to judge for himself which electrodes are closest to the stenosis site as well as the depth of the stenosis for each pair of electrodes. After displaying the graphical images, the treatment control module 54 would then select the proper electrodes pairs to energize and the electrical parameters for the selected pair as the protocol. For example, in FIG. 2B, assume that electrodes starting from the one at 12 o′clock position, clock-wise, are numbered 1 through 8. In that case, the selected electrodes may be pair 0-1, 1-2, 2-3 and 3-4. The selected voltages for the pairs may be 500 V/cm for pairs 0-1 and 3-4, and 1200 V/cm for pairs 1-2 and 2-3. Alternatively, the voltage may be the same for all selected pairs, but the number of pulse repetition may be greater for pairs 1-2 and 2-3 since a larger ablation region can be obtained with a larger number of pulses applied. Alternatively, the pulse duration may be greater for pairs 1-2 and 2-3. The treatment control module 54 then displays on the display device 11 the determined protocol for the user to change or accept. The treatment control module 54 allows the user to change the electrode pairs and other electrical parameters such as voltage, pulse duration and number of repetition for each pair. Preferably, each pulse is a biphasic pulse. To ensure that the thermal damage, if any, is minimized, the treatment control module 54 may apply a few pulses to one pair, apply some pulses to another pair, and then come back to the original pair to apply remaining pulses.

For example, the module 54 may control the switch to electrically energize pair 0-1 for 10 times, 3-4 for 10 times, 1-2 for 10 times, 2-3 for 10 times, and then repeat the same pattern for 10 times for a total number of 100 repetition for each electrode pair.

Even in such an embodiment, it is preferred to have 2-3 radio-opaque markers as well as at least one intravascular ultrasound marker included, especially in cases where a catheter style electrode is used for applications beyond restenosis ablation, such as a minimally invasive method for targeting ablation/Electrochemotherapy/gene transfer in a region of tissue between two vessels. In such a protocol, one catheter style electrode can be inserted into a first vessel and a second catheter style electrode is inserted into a second vessel proximal in location to the first vessel. Targeting the tissue between the vessels for ablation, wire(s) facing each other from each catheter can be energized to target the tissue in this region. Preferably, only the wires facing each other are energized so that surrounding tissue is not affected.

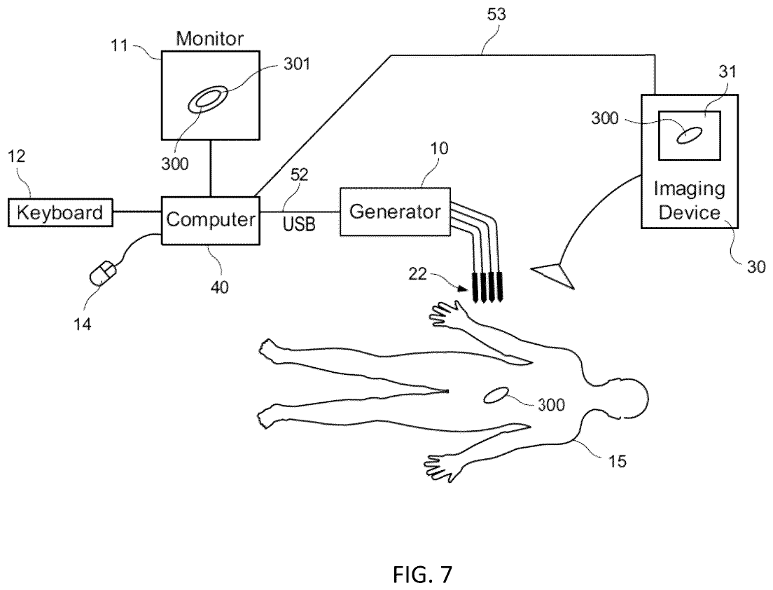

FIG. 7 is a schematic diagram illustrating an IRE system of the invention as disclosed more fully in PCT Patent Application No. PCT/US10/29243, filed Mar. 30, 2010 and entitled “System and Method for Estimating a Treatment Region for a Medical Treatment Device and for Interactively Planning a Treatment of a Patient”, incorporated herein by reference. As illustrated, representative systems can comprise a computer 40 comprising or in operable communication with a computer program embodied in a computer-readable storage medium, which program when executed, enables the computer to operate an IRE medical device. The computer 40 is in operable communication with a mouse 14, keyboard 12, and monitor 11 to enable a user to operate the IRE system. Optionally, computer 40 is in operable communication with one or more imaging modality 30, such as an x-ray, for identifying target tissue in a patient 15 and/or identifying the orientation of electrodes inserted into patient 15. Target tissue 300 (but also inserted electrodes 300) can be viewed on the screen 31 of imaging device 30, as well as on monitor 11. During an IRE procedure, the IRE treatment area 301 can be viewed on monitor 11. Computer 40 is also in operable communication with an electrical pulse generator 10, which in turn is in operable communication with electrodes 22. Any electrical connection between components of the system can be used, as for example a USB connection can be used to connect the computer 40 with electrical charge generator 10.

In specific embodiments, an intravascular IRE system is provided comprising: one or more intravascular catheter type electrode 22 having an inflatable balloon and a plurality of electrically conductive wires disposed lengthwise along the electrode and circumferentially spaced a selected distance from one another; an electrical pulse generator 10 in operable communication with and for delivering electrical pulses to the plurality of electrically conductive wires of the electrodes 22; and a control system 40, 30 in operable communication with the electrical pulse generator 10 comprising a computer program embodied in a computer-readable storage medium, which program when executed, enables a computer 40 to perform a method comprising: determining orientation of the wires of the electrode relative to target tissue 300; selecting one or more but less than all of the electrically conductive wires for administering the electrical pulse(s); and energizing the selected wires to deliver the electrical pulse(s) to the target tissue 300.

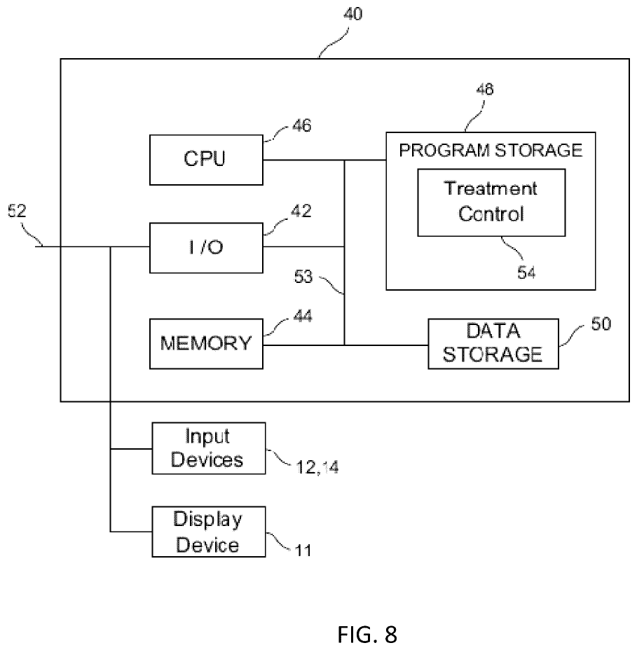

FIG. 8 is a schematic diagram illustrating a control system for implementing methods of the invention and/or operating systems and devices of the invention. Representative embodiments of control systems 40 of the invention can include a computer or computer system with a central processing unit (CPU) 46. The computer system 46 is in operable communication with a power source 52 for supplying electrical power to run the computer, which power supply is controlled using an on/off switch 42. The CPU 46 is operationally connected with one or more computer programs 48 for operating an IRE device or system of the invention. The computer program 48 can comprise instructions 54 for implementing treatment procedures of the invention. By way of connection 53, CPU 46 is in operable communication with memory 44 and one or more data storage device 50. Together, the CPU 46, memory 44, and data storage 50 run computer program(s) 48/54 to operate IRE systems or devices of the invention according to one or more of the methods described in this specification. One or more input devices 12, 14 are in operable communication with the computer system 40 to provide information needed for implementing the treatment protocols. For example, input devices 12, 14 could include one or more imaging modalities to provide information to the practitioner about the target region of interest of a patient, such as shape and size of a tumor or restenosis, or information about the orientation of an electrode in a patient, especially with respect to orientation of certain electrically conductive wires of the electrode relative to a target region of interest. The imaging modalities can include for example MRI, CT, or x-ray. Another such input device 12, 14 could include sensors for collecting information about the tissue being treated, such as current or conductance information. One or more display device 11, such as a monitor, can also be operationally connected with systems of the invention for the practitioner to be able to view the target region of interest and/or positioning or orientation of electrodes in a patient.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. Other embodiments of the invention will be apparent to those skilled in the art from consideration of the specification and practice of the invention.

For example, the device and method described herein may be used to treat other types of lesions such as aneurysm of a blood vessel.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1-34. (canceled)

35. A method for treating a target lesion by irreversible electroporation comprising:
positioning a plurality of elongated electrodes disposed lengthwise and circumferentially spaced a selected distance from one another at the target lesion;
selecting any combination of electrodes among the plurality of electrodes for administering a plurality of electrical pulses; and
activating a generator to administer the plurality of electrical pulses through only the selected electrodes to the target lesion in an amount which is sufficient to induce irreversible electroporation of cells of the target lesion, wherein at least one of the electrical pulses is applied to a first electrode and a second electrode separated by at least one unenergized electrode.

36. The method of claim 35, wherein the plurality of electrical pulses is administered by sequentially activating different pairs of electrodes.

37. The method of claim 35, wherein the plurality of electrical pulses is administered by activating pairs of electrodes positioned in a manner capable of treating the target lesion.

38. The method of claim 35, wherein the plurality of electrical pulses is administered in a manner such that a different voltage is applied to different pairs of electrodes.

39. The method of claim 35, wherein the plurality of electrical pulses is administered in a manner that provides for an electric field distribution ranging from about 5 to 5,000 V/cm.

40. The method of claim 35, wherein the activating of the generator includes controlling a switch to select at least one electrode to which the electrical pulses are not to be applied.

41. The method of claim 35, wherein at least one electrical pulse is applied with a pulse duration of from 1 μs to 1 ms.

42. The method of claim 35, further comprising determining at least one individualized electrical parameter for a pair of electrodes.

43. The method of claim 35, wherein the positioning is determined by applying test pulses and measuring at least one electrical characteristic of tissue cells.

44. The method of claim 43, wherein the positioning includes measuring an electrical resistance as the at least one electrical characteristic of the tissue cells.

45. A method for treating a target lesion by irreversible electroporation comprising:
positioning a plurality of elongated electrodes disposed lengthwise and circumferentially spaced a selected distance from one another at the target lesion; and
activating a generator to administer a plurality of electrical pulses through only selected electrodes to the target lesion in an amount which is sufficient to induce non-thermal ablation of cells of the target lesion,
wherein the selected electrodes includes a number of electrodes less than a total number of electrodes in the plurality of elongated electrodes, and
wherein the selected electrodes includes at least a first electrode and a second electrode separated by at least one unenergized electrode.

46. The method of claim 45, wherein the plurality of electrical pulses is administered by sequentially activating different pairs of electrodes.

47. The method of claim 45, wherein the plurality of electrical pulses is administered by activating pairs of electrodes positioned in a manner capable of treating the target lesion.

48. The method of claim 45, wherein the plurality of electrical pulses is administered in a manner such that a different voltage is applied to different pairs of electrodes.

49. The method of claim 45, wherein the plurality of electrical pulses is administered in a manner that provides for an electric field distribution ranging from about 5 to 5,000 V/cm.

50. The method of claim 45, wherein the activating of the generator includes controlling a switch to select at least one electrode to which the electrical pulses are not to be applied.

51. The method of claim 45, wherein at least one electrical pulse is applied with a pulse duration of from 1 μs to 1 ms.

52. The method of claim 45, further comprising:
determining at least one individualized electrical parameter for a pair of electrodes.

53. The method of claim 45, wherein the positioning is determined by applying test pulses and measuring at least one electrical characteristic of tissue cells.

54. The method of claim 53, wherein the positioning includes measuring an electrical resistance as the at least one electrical characteristic of the tissue cells.
